(19) United States
(12) Patent Application Publication
(10) Pub. No.: US 2021/0213237 A1
(43) Pub. Date: Jul. 15, 2021

(54) DIGITAL AROMA DISPERSION SYSTEM FOR PREDICTING AND MITIGATING MOTION SICKNESS

(71) Applicant: Inhalio, Inc., Scotts Valley, CA (US)

(72) Inventor: Keith Kelsen, Scotts Valley, CA (US)

(21) Appl. No.: 17/059,038

(22) PCT Filed: Aug. 7, 2019
(86) PCT No.: PCT/US2019/045566

§ 371 Date: Nov. 25, 2020

Related U.S. Application Data
(60) Provisional application No. 62/683,238, filed on Jun. 11, 2018.

(51) Int. Cl. A61M 21/00; A61L 9/12

(52) U.S. Cl. CPC A61M 21/00; A61L 9/122; A61L 2209/111; A61M 2209/088; A61M 2205/502; A61M 2021/0016; A61L 2209/133

(57) ABSTRACT

The present invention is directed to a digital aroma system that provides a scented air on demand in a vehicle to prevent or mitigate nausea. Fragrance cartridges are held in a fragrance module having a manifold and valves that are controlled by a processor. The system can open valves to direct an airflow through an anti-nausea fragrance cartridge in response to a user input, detected location of prior nausea, or detected nausea inducing vehicle motions.

### CROSS REFERENCE TO RELATED APPLICATIONS

This patent application claims priority to U.S. Provisional Patent Application No. 62/683,238, “Digital Aroma Dispersion System For Predicting And Mitigating Motion Sickness” filed 11 Jun. 2018, which is hereby incorporated by reference in its entirety.

### BACKGROUND

Motion sickness is a common problem with transportation vehicles such as cars, buses, planes, boats, and trains. Symptoms include fatigue, uneasiness, dizziness, and vomiting. Typical actions that can reduce the symptoms of motion sickness include focusing the eyes on objects straight ahead of the person as well as wrist bands, oral and transdermal medications that can reduce these symptoms.

Various studies show that there is a large percentage of adults will experience car sickness in autonomous cars because the passengers will be multi-tasking while riding in these vehicles. http://umich.edu/˜driving/publications/Motion-Sickness--Report-061616pg-sent.pdf What is needed is an improved fragrance system which can predict and mitigate the symptoms of motion sickness, which can also be integrated into the vehicle or stand alone and does not use scented oils and is not carcinogenic.

### SUMMARY OF THE INVENTION

Motion sickness may be caused by conflicting signals in the inner ear, eyes, and sensory receptors. Motion is sensed by the brain through different pathways of the nervous system including the inner ear, the eyes, and the tissues of the body surface. The present invention is directed to a digital aroma system that provides aroma experiences that can be utilized in vehicles to predict or respond to motion sickness with one or more fragrances which can reduce the symptoms of motion sickness. The inventive anti-nausea fragrance system can be used with any type of moving vehicle including: cars, vans, buses, off-road vehicles, trains, helicopters, airplanes, hovercrafts, hydrofoils, boats, ferries, etc.

The present invention is a digital aroma system that utilizes dry fragrance infused beads or other solid substrate that contain porous fragrance materials contained in a fragrance cartridge(s) that is removable mounted in an interchangeable cassette system that that connects to a manifold. The manifold has specific airway passages that are connected to fans or pumps that are controlled by a computer processor. In response to a fragrance control signal or a fragrance trigger, the processor can selectively direct air into the any individual target fragrance cartridge. More specifically the processor can cause the fan or pump to pull or push fresh unscented air through the target fragrance cartridge and the fresh air passes by the particles infused with a dry fragrance material. The aroma reaches the individual through one or several outlets.

The invention digital aroma system is designed to fit into a very small footprint while providing many aromas that enhance the user's ride experience in the vehicle. In an embodiment the digital aroma system can simultaneously hold numerous (for example six) distinct fragrance cartridges. The digital aroma system can be coupled to or integrated into a vehicle or alternatively, the digital aroma system can be incorporated into a separate device such as a system that clips on the headrest posts, or a headset, an apparatus worn around the neck or a handheld device.

The digital aroma system invention can include a processor that runs computer software that detects vehicle and user movements that may result in motion sickness and creates an anti-nausea smell sensory experience. This computer processor of the digital aroma system can also communicate with remote computers in a cloud-based system and/or a remote server. In an embodiment, the digital aroma system can communicate wirelessly through Blue Tooth, Wi-Fi, RFID or similar technologies with other devices, which can provide control signals or triggers for releasing fragrances.

The digital aroma system can include a processor that can control and monitor the operation of the system components. The processor can be coupled to fans and/or valves to selectively direct air to the target fragrance cartridge. When a desired fragrance signal or trigger is detected, the processor can direct fresh air through the air inlet to the target fragrance cartridge. The dry fragrance can mix with the fresh air and be directed to a scent outlet to the system user. In some embodiments, the processor can direct fresh air through two or more target fragrance cartridges to provide a mixed fragrance to the user. The scent is provided as a limited predetermined period of time or volume of air. Once the scent is provided to the user, the processor can the stop the flow of air through the fragrance cartridge by stopping a fan(s) or closing a valve(s). In an embodiment, the processor can be programmed to flush the scent outlet of the manifold periodically with fresh air so that subsequent fragrances are not mixed or contaminated. For example, the processor may direct fresh air through the scent outlet after each fragrance output by the system.

The digital aroma system can release fragrances based upon control signals or triggers. The digital aroma system can include a receiver, which receives fragrance signals. In response to the fragrance signals, the processor can identify the corresponding target fragrance cartridge and direct air to the target fragrance cartridge, which can result in the dry fragrance device delivering a dry fragrance aroma to the user.

In some embodiments, the digital aroma system can respond to manual inputs. For example, in an embodiment the digital aroma system can have an input which can allow the user to input a level which can range from 1-5 in motion sickness discomfort. The discomfort levels can be: 0 No symptoms, Yawning, Clammy, Lightheaded, 2 Burping, Lethargic, Dizzy, 3 Twisted or upset Stomach, Drowsy, Salivating, 4 Near Vomit, Head Spinning, Exhausted, Disoriented, 5 Vomiting, Head tumbling, Extreme sweating. The user can input the motion sickness discomfort level and the digital aroma system can respond by emitting an anti-nausea fragrance which can vary in volume and fragrance based upon the discomfort level. For example, when the user inputs a discomfort level of 0, the system can emit normal fragrances desired by the user which may not have any anti-nausea properties. In an embodiment, the anti-nausea fragrance is a proprietary blend of dry of at least 2 of the following scents: Peppermint, Spearmint, Lemon, Ginger, and Lavender. The percentages of each scent of the proprietary blend varies on the combination and does not always include all of these ingredients.

When the user inputs a discomfort level of 1, the system can output a small volume of a first anti-nausea fragrance and if the user inputs higher discomfort levels the system can output higher volumes of the first anti-nausea fragrance. In another embodiment, the system can change the anti-nausea fragrance based upon the user input higher discomfort level. When a user inputs a level 1 discomfort level, the system can emit a first anti-nausea fragrance, when the inputs a level 2 discomfort level, the system can emit a second anti-nausea fragrance, when the inputs a level 3 discomfort level, the system can emit a third anti-nausea fragrance, etc.

In an embodiment, the user input can be through wireless communications with a mobile application running on a smart phone. The input can be an electro-mechanical input such as a touch screen input, an audio input such as a microphone, The digital aroma system can provide a feed-back loop. The system can analyze and record the inputs of the user including bio-metric inputs, and the movements of the vehicle with sensors such as accelerometers and gyroscopes. Based upon this information the system can learn the personal preferences of the user based on their feedback experience. The data from the user's discomfort level as well as the vehicle's route mapping and acceleration, deceleration, and G-force can be used by an Artificial Intelligence (AI) predictive system to predict the movements of the vehicle and the discomfort level of the system users.

In some embodiments, the digital aroma system can respond automated triggers. Such as rolling, acceleration, ambient odor, etc. For example, the system may interact with movement or motion sensors to detect and predict vehicle movements. The system can then disperses specific motion sickness reducing scent based on acceleration, deceleration, and G-force in curves. Interacts with google maps or any other mapping system and predicts g-force based on the speed of the vehicle and curves of the road. This will automate the dispersion process using AI to effectively predict motion sickness based on the route mapped, speed and timing of when the event will occur. This will then diffuse the motion scent just prior to the G-force effect. Therefore pre-empting the motion sickness all together. Scents that can reduce motion sickness can include proprietary blends of Peppermint, Spearmint, Lemon, Ginger and Lavender.

### BRIEF DESCRIPTION OF THE DRAWINGS

The present invention will be readily understood by the following detailed description in conjunction with the accompanying drawings, wherein like reference numerals designate like structural elements, and in which:

FIG. 1 illustrates a bottom view of an embodiment of a fragrance cartridge.

FIG. 2 illustrates a bottom perspective view of an embodiment of a fragrance cartridge.

FIG. 3 illustrates a side view of an embodiment of a fragrance cartridge.

FIG. 4 illustrates a bottom perspective view of an embodiment of a fragrance cartridge.

FIG. 5 illustrates a top view of an embodiment of a cassette that holds a plurality of fragrance cartridges.

FIG. 6 illustrates a top perspective view of an embodiment of a cassette.

FIG. 7 illustrates a side view of an embodiment of a cassette.

FIG. 8 illustrates a top perspective view of an embodiment of a cassette with a plurality of fragrance cartridges.

FIG. 9 illustrates a perspective view of an embodiment of a cassette with a plurality of fragrance cartridges.

FIG. 10 illustrates a side view of a car with an integrated digital aroma system with a user interface for a digital aroma system.

FIG. 11 illustrates a front view of an embodiment of a user interface for a digital aroma system.

FIG. 12 illustrates a top view of an embodiment of a digital aroma system.

FIGS. 13 and 14 illustrate bottom views of different embodiments of a vehicle interface with the cassettes removed.

FIG. 15 illustrates an embodiment of a headset that includes an integrated digital aroma system.

FIG. 16 illustrates an embodiment of digital aroma system components for a headset.

FIG. 17 illustrates a side view of a tablet computer with a digital aroma system attached to a back surface.

FIG. 18 illustrates a side view of a smart phone with a digital aroma system attached to a back surface.

FIG. 19 illustrates top cross section view of an embodiment of a fragrance cartridge.

FIG. 20 illustrates top cross section view of an embodiment of a fragrance cartridge.

FIG. 21 illustrates side cross-section view of an embodiment of a fragrance cartridge.

FIG. 22 illustrates side cross-section view of an embodiment of a fragrance cartridge.

FIG. 23 illustrates a bottom perspective view of an embodiment of a fragrance cartridge cassette.

FIG. 23 illustrates a top perspective view of an embodiment of a cassette and a manifold module.

FIG. 24 illustrates a perspective exploded view of an embodiment of a cassette and a manifold module.

FIG. 25 illustrates a top view of an embodiment of a manifold module.

FIG. 26 illustrates a top perspective view of an embodiment of a manifold assembly with cassettes.

FIG. 27 illustrates a top perspective view of an embodiment of a digital aroma system.

FIG. 28 illustrates a top view of airflow through an embodiment of a digital aroma system.

FIG. 29 illustrates a block diagram of components for an embodiment of a digital aroma system.

FIG. 30 illustrates a top view of a multi-seat vehicle with an integrated fragrance system vents and fragrance sensors;

FIG. 31 illustrates a flowchart diagram of a manual nausea input process.

FIG. 32 illustrates a flowchart diagram of a predictive nausea input process.

FIG. 33 illustrates a flowchart diagram of a route predictive nausea input process.

FIG. 34 illustrates diagram of an embodiment of light, scent and audio outputs of an in-vehicle sensory experience network system.

FIG. 35 illustrates a flowchart diagram of a process for fragrance cartridge production and recycling.

FIG. 36 illustrates a computer system, which can be used with a fragrance dispersion system.

### DETAILED DESCRIPTION

A detailed description of one or more embodiments of the invention is provided below along with accompanying figures that illustrate the principles of the invention. While the invention is described in conjunction with such embodiment(s), it should be understood that the invention is not limited to any one embodiment. On the contrary, the scope of the invention is limited only by the claims and the invention encompasses numerous alternatives, modifications, and equivalents. For the purpose of example, numerous specific details are set forth in the following description in order to provide a thorough understanding of the present invention. These details are provided for the purpose of example, and the present invention may be practiced according to the claims without some or all of these specific details. For the purpose of clarity, technical material that is known in the technical fields related to the invention has not been described in detail so that the present invention is not unnecessarily obscured.

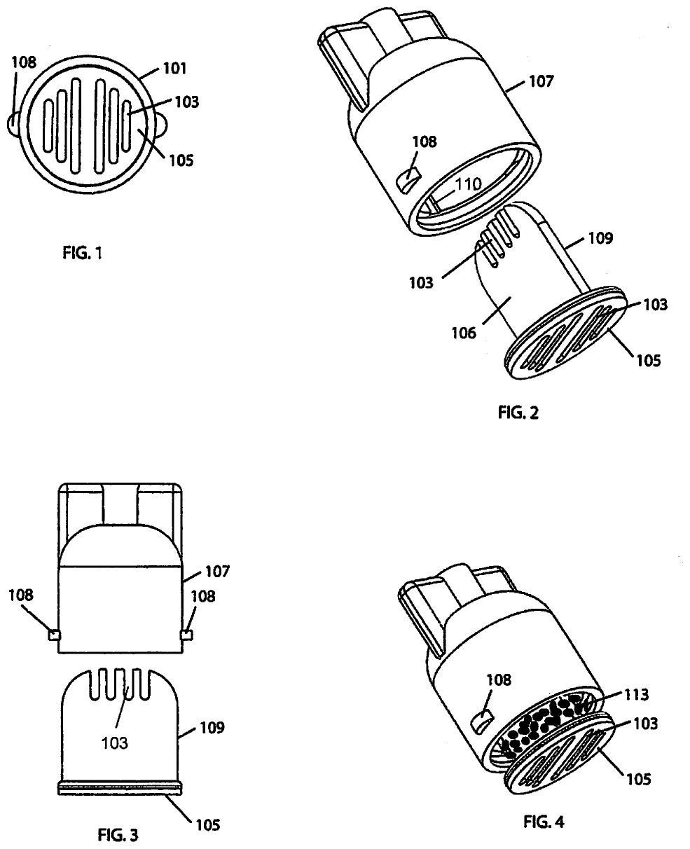

An embodiment of a fragrance cartridge is illustrated in FIGS. 1-4. FIG. 1 illustrates a bottom view of an embodiment of the fragrance cartridge 101 with a plurality of airflow slots 103 in the bottom surface 105. In an embodiment tabs 108 can be mounted on the outer surface of the cartridge 101 which are used to secure the cartridge to a cassette. FIG. 2 illustrates a perspective view of the fragrance cartridge 101 in a disassembled state. In this embodiment the fragrance cartridge 101 includes an upper housing 107 structure which has an internal volume and a lower housing 109 structure which has a lower surface 105 and a center divider 106 having air flow slots 103. The center divider 106 can have an outer surface which fits within a groove 110 formed in the internal surface of the upper housing 107. FIG. 3 illustrates a side view of a fragrance cartridge 101 that has a two piece housing that includes an upper housing 107 and a lower housing 109 that are secured together to form the complete housing for the fragrance cartridge 101. FIG. 4 illustrates a perspective view of the fragrance cartridge 101 in a disassembled state. The upper housing 107 can be filled with a plurality of substrates 113 that are infused with a dry fragrance. In an embodiment the substrates 113 can be spherical balls or other three-dimensional objects such as cubes, cylinders, particles or other geometric volumes before the outer diameter of the bottom surface 105 structure can be fused or coupled to the inner diameter of the bottom of the upper housing 107 structure.

While the fragrance cartridge 101 is illustrated as a dome shape with slots 103 in the lower surface 105 and the lower surface 109, in other embodiments the fragrance cartridge can have any other geometric shape that can hold the plurality of substrates 113. The width of the substrates 113 is wider than the slots 103 in the lower surface 105 and a center divider 106. When air flows through the cartridge 101, the dry fragrance can mix with the air and be removed from the substrates 113 resulting in scented air exiting the cartridge 101. In an embodiment, the lower portion of the fragrance cartridge 101 can have a cylindrical shape that can be placed into a corresponding cylindrical bore in a manifold or cassette matrix.

In an embodiment with reference to FIGS. 5-8 an embodiment of a fragrance cassette matrix 115 is illustrated. The illustrated embodiment of the cassette matrix 115 can have five cartridge sockets 117 formed in the upper surface that securely hold five fragrance cartridges 101 in a single row configuration. In other embodiments, the cassette matrix can hold more fragrance cartridges 101 in different configurations such as a 2×6, 3×8 or any other one or two or three dimensional array configuration including circular cassette matrix configuration.

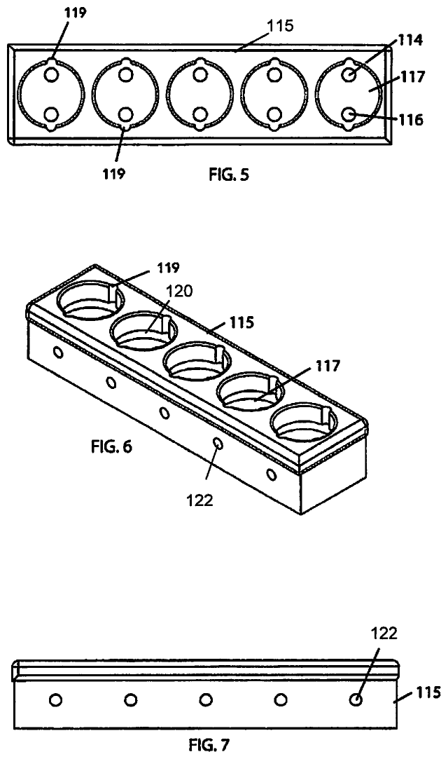

Each of the cartridge sockets 117 has two air channels, one inlet 114 and one outlet 116 and can be keyed with tab slots 119 so that fragrance cartridges 101 can be easily placed in and removed from the sockets 117. FIG. 5 illustrates a top view of a fragrance cassette matrix 115. FIG. 6 illustrates a perspective top view of the cassette matrix 115. The cassette matrix 115 can have cartridge openings 117 that each holds a fragrance cartridge. FIG. 7 illustrates a side view of the cassette matrix 115. The cassette matrix 115 can have inlet holes formed on one side of the cassette matrix 115 and outlet holes 122 formed on the opposite side of the cassette matrix 115. The inlet holes can be coupled to the inlet holes 114 and the outlet holes 122 can be coupled to the outlets 116.

The cylindrical cartridge openings 117 can be aligned with the lower cylindrical cartridge and the tab slots 119 can be aligned with the tabs on the side of the lower cylindrical cartridge. The tab slots 119 can be vertical slots extend down the sides of the cartridge openings 117. The tab slots 119 can intersect circular slots 120 which extend around a lower inner diameter portion of the cartridge openings 117. Once the fragrance cartridge is fully inserted into the cartridge socket 117, the fragrance cartridge can be axially rotated within the socket 117 so that the tabs are in the circular slots 120 and are no longer aligned with the tab slots 119. By offsetting the tabs from the tab slots 119, the fragrance cartridge can be secured within the cassette matrix 115. The tab and tab slots 119 can provide a mechanism for securing the fragrance cartridges to the cartridge sockets 117. When the fragrance cartridge is placed in the cartridge socket 117, the cartridge tabs can be placed in the tab slots 119.

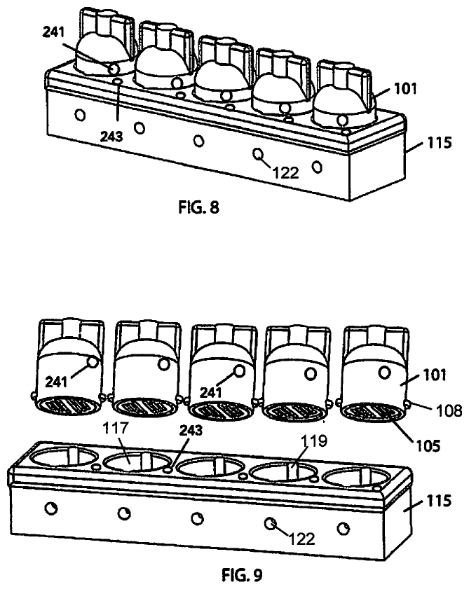

FIG. 8 illustrates a perspective top view of the cassette matrix 115 with the fragrance cartridges 101 positioned in the cartridge sockets 117. In the illustrated embodiment, the cartridges 101 have been inserted into the cartridge sockets 117 with the tabs aligned with the tab slots and then rotated 90 degrees after being fully inserted. FIG. 9 illustrates a perspective side view of the cassette matrix 115 and with the cartridges 101 positioned over the socket openings 117. When the cartridges 101 are inserted into the socket openings 117 the tabs 108 are aligned with the tab slots 119. The cartridges 101 are interchangeable within the cassette matrix 115. In the fully inserted position, the inlet holes 114 and the outlet holes 116 at the bottom of each cartridge socket 117 are adjacent to the slots (103 FIG. 1) formed with the bottom of the cartridge with the center divider (106 FIG. 2) between the inlet holes 114 and the outlet holes 116.

As discussed each cartridge 101 can include identification information which identifies the fragrance so that the digital aroma system can properly direct air to the target fragrance cartridge 101 regardless of its position in the cassette matrix. For example, in an embodiment, each fragrance cartridge 101 can include a radio frequency identification (RFID) tag 241 and the cassette matrix 115 can include RFID readers. The RFID tag 241 can transmit fragrance identification and a number of fragrance dispersions and a cartridge identification code. The RFID reader 243 can read the identification information from the RFID tag 241 on the fragrance cartridge 101 and additional cartridge information, which can be used by the system. For example, the system display the fragrance on a system output and direct the air to the proper fragrance cartridge 101.

The cassette with fragrance cartridges can be used with various digital aroma system assemblies. FIG. 10 illustrates a side view of an embodiment of a car 306 with an integrated digital fragrance system 121 and a user interface 122. The digital fragrance system 121 can be integrated within the dashboard area of the car 306. The user interface 122 can be an input device with a visual display output such as a touch screen or a visual display with input buttons. In other embodiments, the user interface 122 can be displayed on a mobile computing device such as a smart phone or tablet computer which is in wired or wireless communication with the digital fragrance system 121.

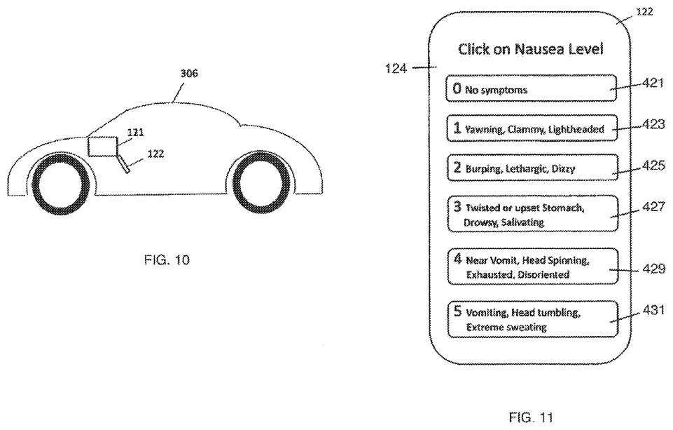

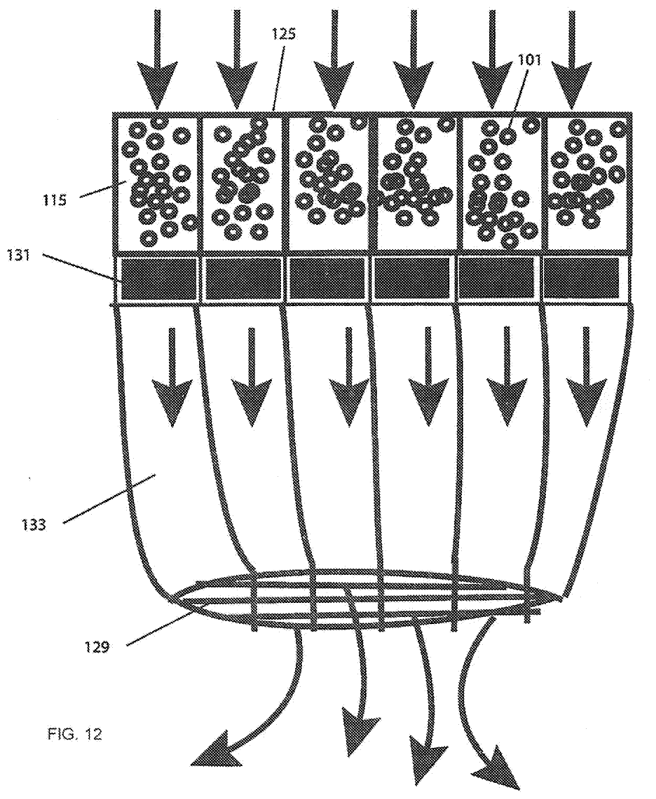

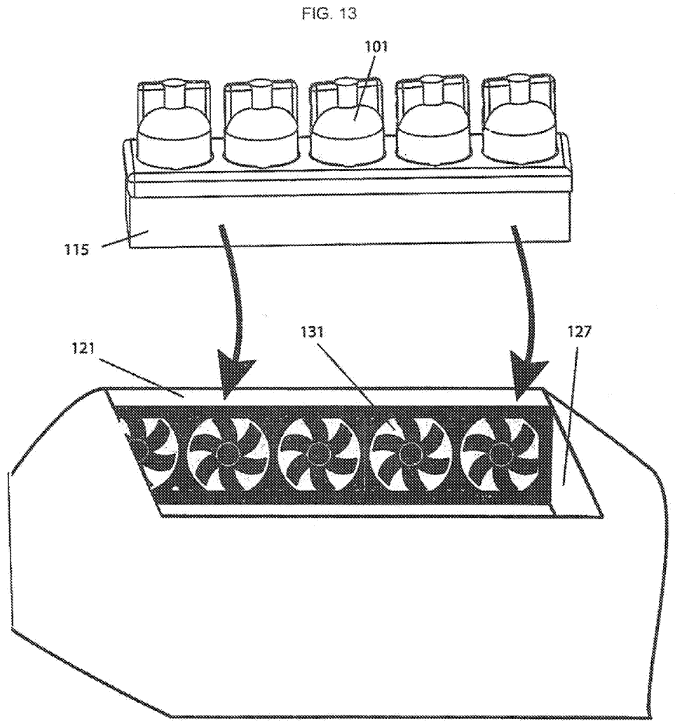

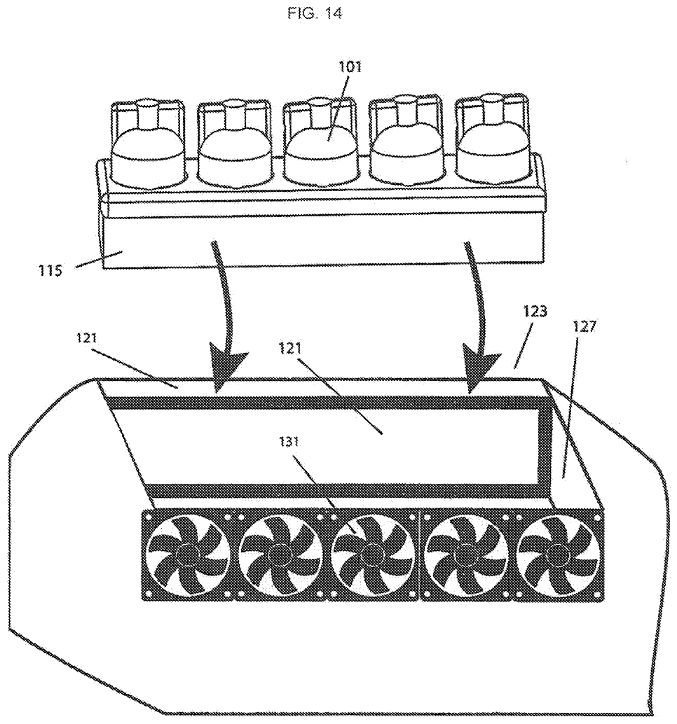

FIG. 11 illustrates a view of a user interface 122 which includes an input of the digital fragrance system. In an embodiment the user interface 122 can be displayed on a touch screen 124 which can communicate with the digital fragrance system. In this example, the user interface 122 can display inputs for nausea level and a passenger can press a button that corresponds to the current or anticipated nausea level. The user interface 122 can switch the visual display to ask the passenger's nausea level periodically or in response to ride conditions such as winding roads which can result in nausea. The passenger can indicate the nausea level by pressing a corresponding nausea level button. The user interface 122 can transmit nausea signals to the digital fragrance system which can respond by emitting anti-nausea fragrances which can be proportional to the nausea level.

In the illustrated example, the user interface 122 can have nausea level inputs that range from: 0 to 5. However, in other embodiments, the nausea level can have any other range of levels. At nausea level 0 there are no symptoms 421 and at nausea level 1 the passenger can start yawning and have clammy palms and be lightheaded 423. At nausea level 2, the passenger can start burping, become lethargic and dizzy 425. At nausea level 3, the passenger can feel like the stomach is twisted or upset, the passenger can feel drowsy and start salivating 427. At nausea level 4, the passenger can be near vomiting and feel like the head is spinning, exhausted and disoriented 429. At nausea level 5, the passenger can start vomiting, feel like the head is tumbling and experiencing extreme sweating 431. In this example, if the user touched the nausea level 0, the digital fragrance system will maintain its current operation and not emit any anti-nausea fragrances.

In other embodiments, the user interface 122 can emit an audio output which asks the passenger what their nausea level is and the passengers' response can be detected by a microphone which can detect an voice input from the passenger(s). The user interface 122 can periodically turn down any audio programs such as music and ask the passenger what their nausea level is. The passenger can indicate their nausea level and the user interface 122 can interpret the passenger's voice and determine the passenger's nausea level. The user interface 122 can transmit nausea signals to the digital fragrance system which can respond by emitting anti-nausea fragrances which can be proportional to the nausea level.

FIG. 12 illustrates an embodiment of the digital aroma system 123 used with vehicle that shows the airflow paths through the system components. The cassette 115 with the fragrance cartridges 101 can be mounted adjacent to the air inlet 125. The fragrance cartridges 101 can each be filled with substrates 113 which are infused with dry fragrances. Micro fans 131 that are individually controlled can be mounted in the digital aroma system 123 adjacent to the cassette 115. The micro fans 131 can be coupled to a processor that selectively actuates the micro fans 131 and directs scented air into a manifold 133 which can include a separate air flow path or channel for each fragrance cartridge 115. By having separate air flow paths for each fragrance cartridge in the manifold 133, there is no contamination and/or mixing of the different scents from the fragrance cartridges 101. The scented air exits the air outlet 129 and is directed towards the user holder of the controller 121. In this configuration, the micro fans 131 create a low gas pressure, which pulls air through the fragrance cartridges 101. In an embodiment, the micro fans 131 can be placed at the scented air outlet 129 so that the manifold 133 is between the cassette and micro fans 131. In other embodiments the micro fans can be positioned before the cassettes to create higher gas pressure that push air through the fragrance cartridges 101. Thus, the micro fans 131 can be placed in various different positions that creates a vacuum and sucks the air through the cartridges 101 and then pushes the air through the manifold. In different embodiments the fans 131 can be replaced by micro pumps

FIG. 13 illustrates a view of an embodiment of an interior surface 121 of a vehicle with an integrated digital aroma system 123 that includes a cassette slot 127 and micro fans 131. To use the digital aroma system 123 the cassette 113 filled with fragrance cartridges 101 can be inserted into the cassette slot 127. The fans 131 are placed forward of the cassette 113 and the manifold. When the digital aroma system 123 is actuated to release a scent, one (or more) of the fans 131 is actuated that creates high pressure that pushes air through the cartridge 101 containing the designated fragrance. The airflow generated by the fans 131 blows scented air through the manifold towards the user of the controller 121.

FIG. 14 illustrates a view of another embodiment of an interior surface 121 of a vehicle. In this embodiment, the micro fans 131 are mounted in a downstream position relative to the cassette slot 127. In this configuration, the cassette slot 127 can be adjacent to the air inlet 125. When the digital aroma system 123 is actuated to release a scent, one of the fans 131 is actuated creating a vacuum that pulls air through the cartridge 101 containing the designated fragrance to blow scented air through the manifold towards the user of the controller 121.

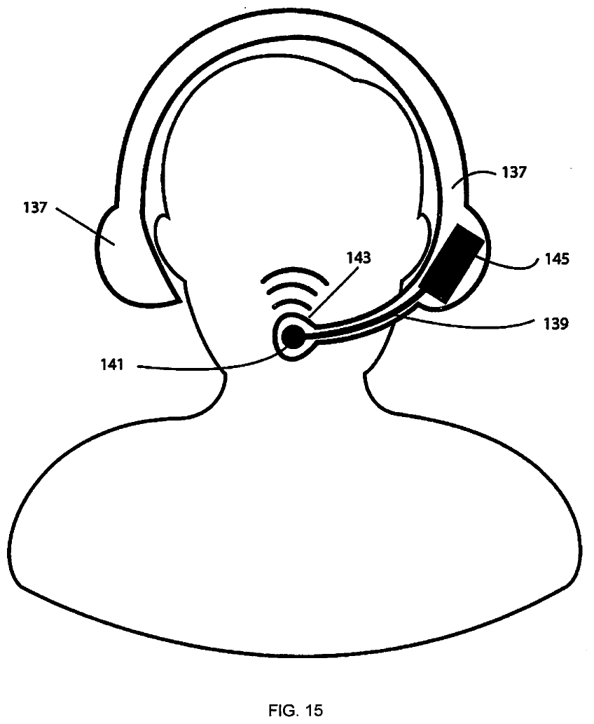

In an embodiment, the anti-nausea digital aroma system can be used as a separate unit worn by a passenger riding in a vehicle. FIG. 15 illustrates a headset 135 that includes an integrated digital aroma system 123. In this embodiment headset 135 that includes ear cups 137 which can include speakers that emit sound and a mouthpiece 143 that includes a microphone that is used for receiving voice commands. In the illustrated configuration, most of the digital aroma system 145 components including the cassette with a replaceable anti-nausea fragrance cartridge, fans and check valves can be mounted in one of the ear cups 137. An air passageway 139 can be built into an arm, which extends from the ear cup 137 to the mouth. When the digital aroma system 145 is actuated to release a scent, one of the fans is actuated that directs air through the fragrance cartridge and blows the scented air through the air passageway towards the nose of the user wearing the headset 135. In an embodiment, the user can tell the system the nausea level with verbal inputs such as “level 2”, “level 5”, “emit maximum anti-nausea fragrance please!”, etc. The system can interpret the use's audio inputs and the system can emit a corresponding fragrance. This system can be particularly useful for passengers who tend to get motion sick. The system may also emit audio signals which can help to comfort the system user. For example, the system may have default audio outputs based upon the user's input nausea level. In an embodiment, the user can configure the system to output audio signals such as relaxing music or binaural tones Binaural beats therapy is an emerging form of soundwave therapy in which the right and left ears listen to two slightly different frequency tones yet perceive the tone as one. The binaural auditory beat that a person hears is the difference in frequency between the left and the right ear and should be at frequencies lower than 1,000 hertz (Hz) for the brain to detect the binaural beat. For example, if the left ear registers a tone at 200 Hz and the right at 210 Hz, the binaural beat heard is the difference between the two frequencies −10 Hz.

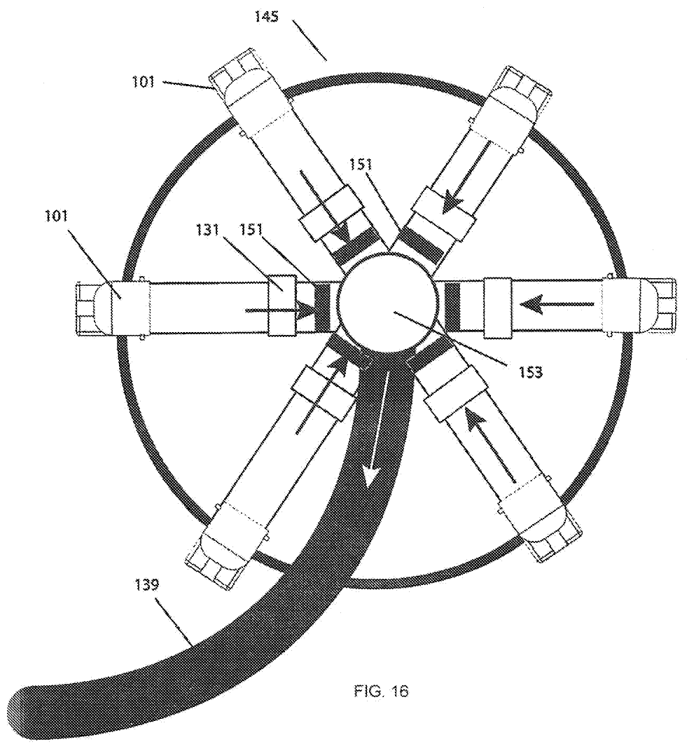

Details of the configuration of an embodiment of the digital aroma system 145 used with the headset system are shown in FIG. 16. In this embodiment, the digital aroma system 145 can include fragrance cartridges 101 which can be individually placed into cartridge holes 147 around the outer surface of the ear cups of the headset with the inlet sides of the cartridges 101 exposed to ambient air. Thus, in this embodiment the digital aroma system 145 may not include a cassette. The user can easily access and exchange each of the individual fragrance cartridges 101. Air passageways 139 can connect each of the fragrance cartridges 101 to a corresponding fan 131 which can blow the scented air from the outer edge of the ear cup 137 into an inner circle 149 and then through an air passageway 139 to the nozzle outlet 141. When a fragrance is to be delivered to a user, a micro fan 131 is actuated which draws air into the interior volume 153 of an ear cup 137 of the headset 135. This air movement pulls fresh ambient air through the fragrance cartridge 101. The scented air is then directed through the air passageway 139 and out an outlet nozzle adjacent to the face of the headset wearer. Also located with the within the passageways of the digital aroma system 145 are small check valves 151 that prevent the back flow of scented air into the other fragrance pathways and keeps the fragrance cartridges 101 and fragrance infused substrates pure and discreet from fragrance contamination.

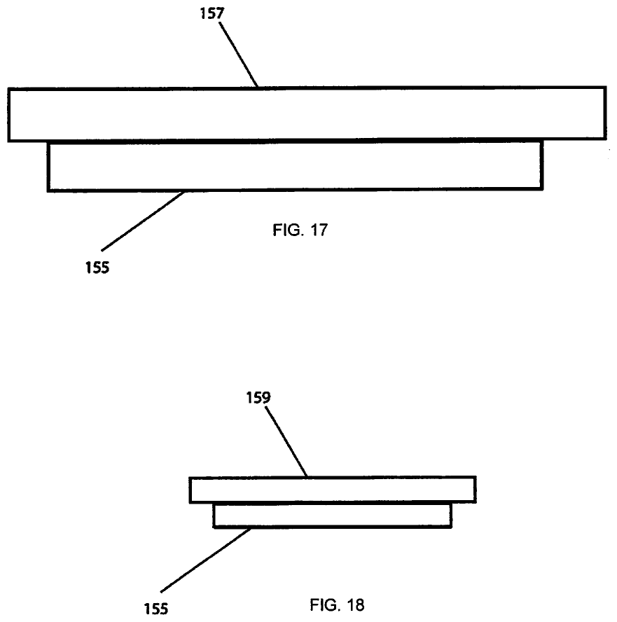

In other embodiments, the digital fragrance system can be attached to various other portable computing devices such as tablet and smart phones. FIG. 17 illustrates a side view of a digital aroma system 155 attached to a back surface of a tablet computer 157 and FIG. 18 illustrates a side view of a digital aroma system 155 attached to a back surface of a smartphone computer 159. In this embodiment, the digital fragrance system can also be independent of the vehicle. The mobile computing device can have integrated input such as a microphone and touch screen. In a basic form of operation, the system can run a mobile application that respond to user inputs and emit a volume of an anti-nausea fragrance that is proportional to the nausea level. The mobile computing device can have motion sensors such as accelerometers, gyroscopes, GPS, maps, cameras, etc. which can interact with the mobile application to predict possible nausea conditions such as high-speed rotation, high centripetal forces, windy roads, traffic, acceleration/deceleration forces etc. The mobile application can detect rotation and/or centripetal forces above a predetermined level and the duration of the rotation or centripetal forces and respond by emitting the anti-nausea.

The volume of anti-nausea fragrance emitted by the system when nausea is likely to occur can be proportional to the intensity of the rotation or acceleration and the duration of the rotation or acceleration. For example, if the system detects a centripetal force of 0.05-0.1 G for an period of time between 30 seconds and one minute, the system can respond by emitting a level 1 volume of anti-nausea fragrance. If the system detects a centripetal force of 0.1-0.2 G for an period of time between one minute and two minutes, the system can respond by emitting a level 2 volume of anti-nausea fragrance. If the system detects a centripetal force of 0.2-0.3 G for an period of time between two minute and five minutes, the system can respond by emitting a level 3 volume of anti-nausea fragrance. The system can escalate the volume of anti-nausea fragrance with higher rotation or acceleration forces and the durations of the rotation or acceleration.

Some studies have shown that humans are more susceptible specific frequencies of wave motion. For example, when test subjects were exposed to a series of different periods of up and down constant velocity motions including 0.2 seconds, 0.7 seconds 1.1 seconds and 1.6 seconds. The test results shows that short duration motions results in very little motion sickness. Motions that lasted 0.7 or 1.6 seconds resulted in more motion sickness and motions that lasted 1.1 seconds produced the most motion sickness in the test subjects. In an embodiment, the system can determine the frequencies of the motions that the user's indicate motion sickness as described above. The system can then predict the likelihood of motion sickness based upon the detected and/or predicted frequencies of the traveling vehicle.

The digital aroma system 155 can include interchangeable fragrance cartridges that are removably attached to a cassette and a manifold that can include fans or pumps and check valves. The digital aroma system 155 can be configured to direct scented air to an air outlet adjacent to the bottom edge of the tablet computer 157 or smart phone 159. However, tablet computers 157 and smart phones 159 can detect the orientation of the screen with accelerometers and adjust the displayed images so that they are always upright. In some embodiments, the digital aroma system 155 can adjust the air output to always be at emitting scented air from the bottom edge of the computing device regardless of the orientation of the digital aroma system 155. In an embodiment the digital aroma system 155 may include fans that normally pull the fresh air through the fragrance cartridges and out the bottom edge of the tablet computer 157. However, the digital aroma system 155 can also detect when the tablet computer 157 or smart phone 159 has been turned upside down. When this orientation change is detected, the fans can be controlled to operate in reverse the airflow to push fresh air from the new top through to the new bottom of the table computer 157.

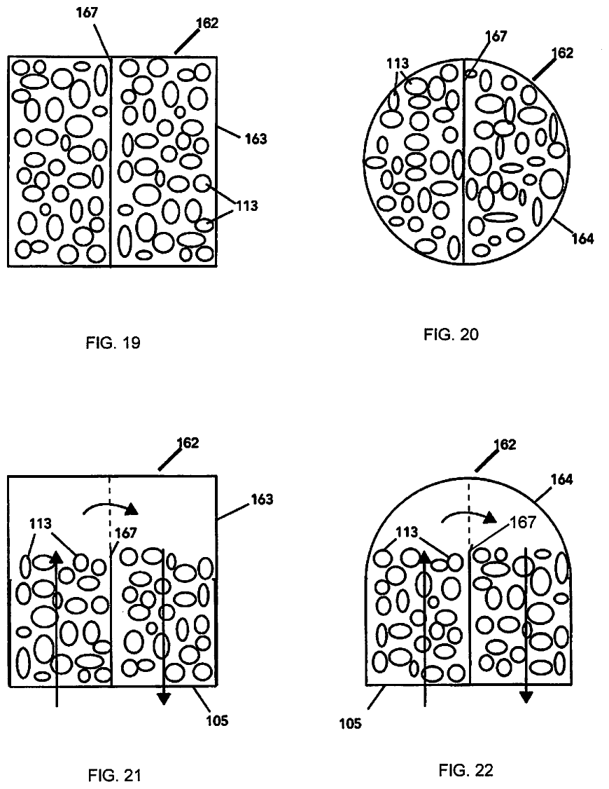

In different embodiments, the fragrance cartridges used with the digital aroma system can be configured with an air inlet and a scented air outlet on the same side of the fragrance cartridge. With reference to FIG. 19 is a top cross section view of a generally cube shaped housing 163 embodiment of a fragrance cartridge 162, which is at least partially filled with fragrance infused substrates 113. The fragrance cartridge 162 includes divider 167 that extends across a center the width of the housing 163. FIG. 20 illustrates a top cross section view of the bullet shaped housing 164 embodiment of a fragrance cartridge 162 which has a lower cylindrical portion and an upper hemispherical portion. The fragrance cartridge 162 can have a divider 167 that extends across the width of the housing 163.

FIG. 21 illustrates a side cross section view of the generally cube shaped housing 163 embodiment of a fragrance cartridge 162 with arrows illustrating the flow path of air through air inlet holes in the lower surface 105 of the housing 163 where the air mixes with the dry fragrance substrates 113. Fragrance particles mix with the air and travel upward and through small holes in the upper portion of the divider 167 which can be planar and rectangular in shape. The fragrance substrates 113 can be larger than the openings in the divider 167 so that the fragrance substrates 113 will not travel from the inlet side of the fragrance cartridge 162 to the outlet side of the fragrance cartridge 162. The air then travels back down on the opposite side of the fragrance cartridge 162 and out through air outlet holes in the lower surface 105 of the housing 163.

In the embodiment shown in FIG. 22, the fragrance cartridge 164 can have a bullet shaped housing with a lower cylindrical shaped housing and an upper half spherical shaped housing. The divider 167 is positioned against the lower surface of the housing 165 and provides a passageway above the divider 167. The arrows illustrating the flow path of air through air inlet holes in the bottom of the lower surface 105, the air mixes with the fragrance substrates 113 and travels up one side the divider and through slots in the upper portion of the divider 167. The air then travels down the opposite side of the fragrance cartridge 164 mixing with the fragrance substrates 113 and travels back through air outlet holes in the in the lower surface 105 of the housing 164.

The fragrance from the substrates 113 that is mixed with the fresh air and emitted by the fragrance system can include different fragrance components including: top notes, middle notes and base notes. Top notes can contain the smallest fragrance molecules, which can quickly dissipate. Middle notes can contain medium sized fragrance molecules that can last longer than the top notes and may dissipate slower than the top note molecules. Base notes can dissipate the slowest and can contain larger fragrance molecules that can last for longer than the middle notes. Base fragrance molecules are larger than middle note fragrance molecules, which are larger than top note fragrance molecules.

As discussed, the fragrance cartridges 162 can have sealed housings that can contain dry fragrance beads and additional air space within the cartridge housing. Rather than completely filling the inner volume of the fragrance cartridges 162 with substrates 113, these sealed cartridge designs have additional air space so that the base notes of the fragrance will infuse into the air molecules residing in the cartridges 162 before each diffusion so that the based notes will be deployed into the air when the airflow starts. Because of the larger mass of the base note fragrance molecules compared to the top and middle note molecules, base note fragrance molecules require more time to be infused into the air inside the cartridges 162. If the cartridges 162 are completely filled with dry fragrance bead substrates 113, then the base note fragrance molecules s will not disperse properly into the cartridge airspace. The volume ratio of air space to dry fragrance beads for optimum base note fragrance molecule infusion can depend upon the type of fragrance. In an embodiment where the fragrance cartridges is a citrus scent, the bead to air ratio can be 75% or 70% to 80% of the volume and the remaining volume occupied by air space. In contrast a denser or heavier fragrance such as tobacco may only require up to 25% or 20% to 30% dry fragrance beads substrates 113 by volume and the remaining volume of air. These dry fragrance bead to air volume ratios can be required to ensure the proper deployment of based note fragrance molecules such as a tobacco scent infusion of the air in the cartridges 162 before the scented air flow is transmitted through the cartridges 162.

Because the cartridges have air inlets and scented air outlets on the same lower surface, the cartridges can be mounted in a cassette that holds the cartridges against a manifold that has both air inlets and scented air outlet paths. FIG. 23 illustrates a bottom perspective view of an embodiment of a cassette 169 that has openings 171 that hold the individual fragrance cartridges 162. The fragrance cartridges 162 be inserted or replaced from the cassette 169. As discussed above, the fresh unscented air inlet 173 and the scented air outlet 175 of the fragrance cartridge 162 can be on the same planar side surface of the cartridge 162. Thus, the cassette 169 can be closed on all but one side since air does not flow through the cassette 169.

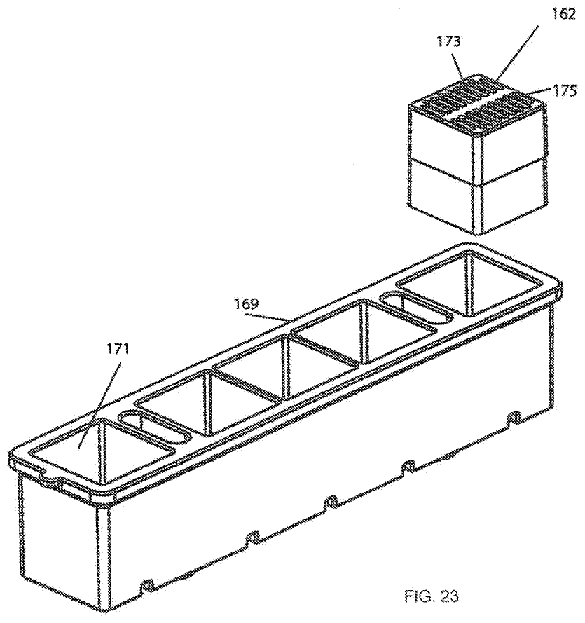

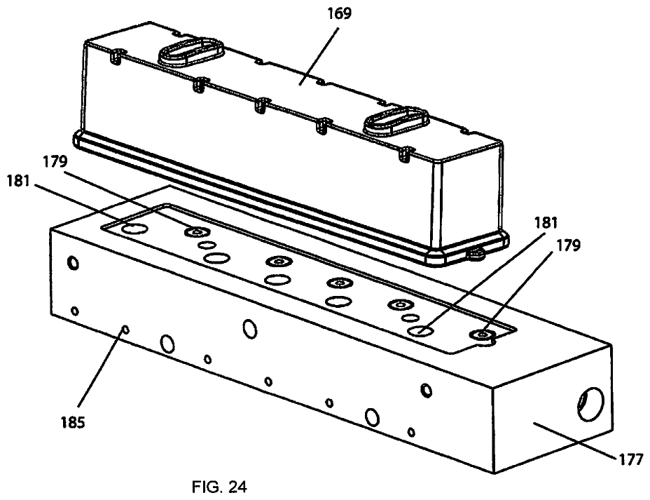

With reference to FIG. 24, a perspective view of an embodiment of the cassette 169 and a manifold module 177 is illustrated. The cassette 169 is in the upright position, which shows the solid upper surface. The air inlet and scented air outlets of fragrance cartridges are exposed on the lower surface of the cassette 169. The manifold module 177 can have a recess 183 that corresponds with the outer perimeter of the cassette 169. The manifold module 177 can also have internal air passageways that are connected to the fragrance cartridges. In this embodiment, the manifold module has a row of fresh air outlet holes 179 and a row of scented air inlet holes 181. The cassette 169 can be placed in the recess 183 and held against the manifold module 177 with a releasable coupling mechanism. A gas seal such as an airtight gasket can be placed between the fragrance cartridges and the manifold module 177 to separate the different fragrance cartridges and seal the fresh air outlet holes 179 and air inlet holes 181. The side surfaces of the manifold module 177 can have side holes 185, which can be connected to the internal passageways within the manifold modules 177 and the fresh air outlet holes 179 and air inlet holes 181.

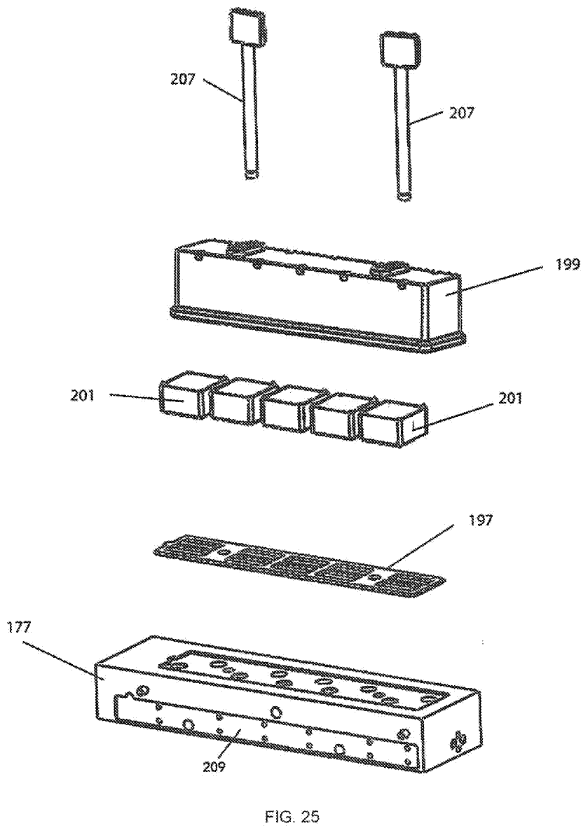

With reference to FIG. 25, an exploded view of a different embodiment of a manifold module 177 and cassette assembly is illustrated. In this embodiment, the assembly can include a cassette chamber 199 that surrounds a plurality of fragrance cartridges 201. Different fragrance infused substrates can be placed in each of the fragrance cartridges 201 that are within the cassette chamber 199. A cassette gasket seal 197 can be placed between the cassette chamber 199 and the manifold module 177 to prevent air from flowing between the different fragrance cartridges 201 or out the top and sides of the cassette chamber 199. The cassette assembly including the cassette chamber 199 and fragrance cartridges 201 are held to the manifold module 177 with locking pins 207 that extend through the cassette assembly components. The locking pins 207 can have threaded ends which can be rotated and tightened into the manifold module 177 to compresses the gasket 197 between the cassette chamber 199 and the manifold module 177, which creates an airtight assembly. When the adjacent manifold modules 177 are attached to each other, a manifold gasket 209 can be placed between the manifold modules 177 to create an airtight seals for the aligned and coupled side air holes.

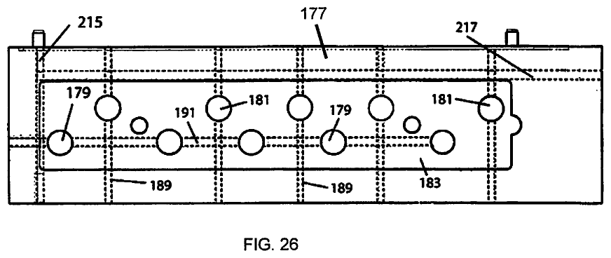

With reference to FIG. 26 a top view of an embodiment of a manifold module 177 which shows the internal passageways which include a length passageway 191 that is connected to the fresh air outlets 179 that extends along the length of the manifold module 177. The internal passageways also include parallel width passageways 189 that extend across the width of the manifold module 177 where each of the width passageways 189 are coupled to a scented air inlet 181. The length passageway 191 is offset vertically from the width passageways 189 so that they are not connected. The manifold module 177 can also include an inlet air passageway 215 that extends through the width of module 177 on one edge and an outlet scent passageway 217 that extends along the length of the module 177 on another edge. An inlet valves (not shown) can be coupled to the length passageway 191 and outlet valves can be coupled to the width passageways. When actuated to open the inlet valve can connect the length passageway 191 to the inlet air passageway 215 and the outlet valves can connect the width passageways to the outlet scent passageway 217. When multiple modules 177 are connected, the inlet air passageways 215 can be connected to form a longer inlet air passageway that extends across the entire width of the assembly. In contrast, when multiple modules 177 are connected, the system may only use the outlet scent passageway 217 of the end module 177 with the outlet scent passageways 217 of the other modules 177 being unused.

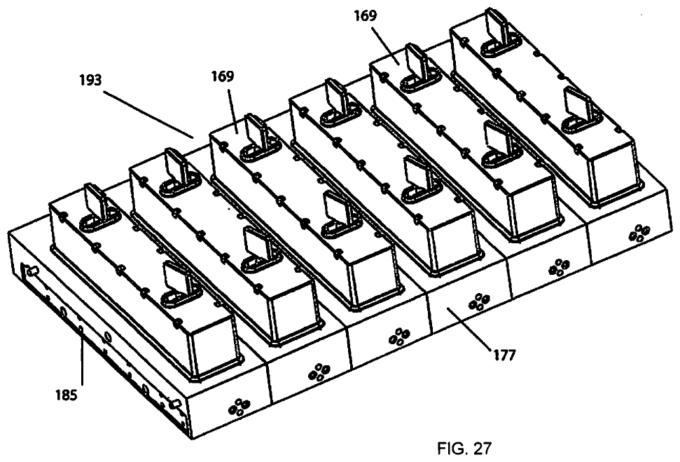

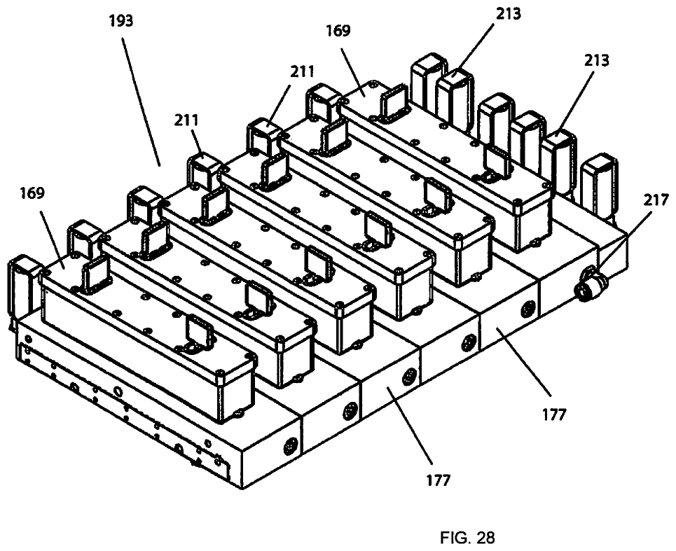

With reference to FIG. 27, a digital aroma system 193 having multiple manifold cassettes 169 mounted on modules 177 can be coupled together with the side holes 185 aligned to form a larger digital fragrance system. By connecting and sealing the side holes 185 to the side holes 185 of the adjacent manifold module 177, the digital fragrance system can be expanded to include any number of fragrance cartridges. In the illustrated example, there are six manifold modules 177 with each of the manifold modules 177 containing five fragrance cartridges. In this example, the illustrated digital aroma system assembly 193 can include a total of thirty fragrance cartridges.

With reference to FIG. 28, the digital aroma system 193 can have a plurality of inlet valves 211 can be coupled to the inlet air passageways on one end of each of the manifold modules 177. A plurality of outlet valves 213 can be coupled to the outlet scent passageways on one of the end manifold modules 177 and the opposite ends of the outlet scent passageways can be sealed to prevent air from escaping. Air can be directed through the digital fragrance system to any individual fragrance cartridge by controlling the open/closed positions of the inlet valves 211 and the outlet valves 213. The digital aroma system 193 formed from a plurality of manifold modules can have an array of internal passageways which can be coupled to fresh air inlets 179, scented air outlets 181, inlet valves 211 and outlet valves 213. The inlet valves 211 and outlet valves 213 are opened and closed to control the scented air outlet path. By actuating (opening) one inlet valve 211 and one outlet valve 213 and keeping all other inlet valves 211 and outlet valves 213 closed, a passageway to a specific fragrance cartridge can be selected by the digital aroma system.

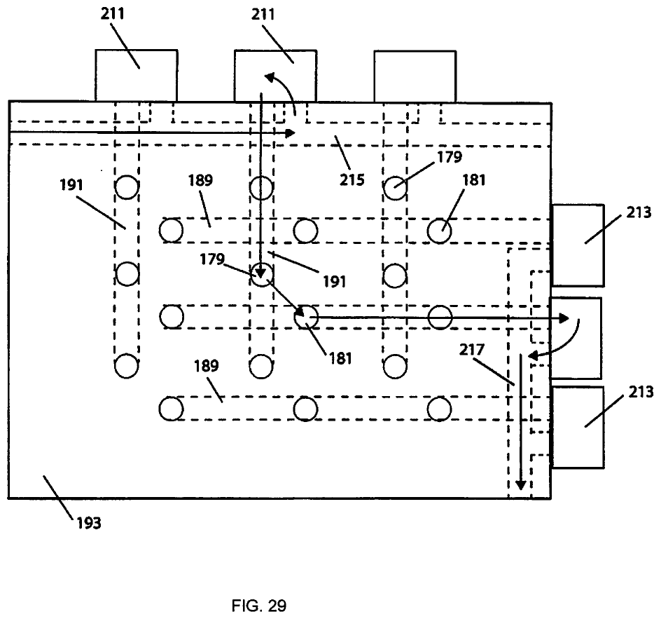

FIG. 29 illustrates a top view of a simplified embodiment of a digital aroma system 193 configured with nine fragrance cartridges spaces for clarity. Each cartridge space includes a fresh air inlet 179 and a scented air outlet 181. In an embodiment pressurized air from a fan or pump can be applied to the inlet air passageway 215. When one of the inlet valves 211 is actuated pressurized air can flow through the corresponding length passageway on a selected row of fragrance cartridges on a single cassette. When one of the outlet valves 213 is open, air can flow through the fragrance cartridges and scented air can flow to the outlet passageway 217. From the simplified digital aroma system 193, the scented air can be directed towards the nose of the system user. In an alternative embodiment a vacuum or low pressure from a fan or pump can be applied to the outlet scent passageway 217. When one of the inlet valves 211 is open, air can be drawn or pulled through the corresponding length passageway on a manifold module 177. Air can then flow through one of the fragrance cartridges to the outlet passageway 217 through the fan or pump and be directed towards the nose of the system user. The valves can be actuated by a valve controller(s) that is controlled by a system processor in response to a scent release signal or trigger. Each individual fragrance stored in the digital aroma system 193 can be output by actuating a combination of one inlet valve and/or one outlet valve. In some embodiments, it can be desirable to mix a plurality of fragrances, which can be performed by opening valves to a plurality of fragrance cartridges.

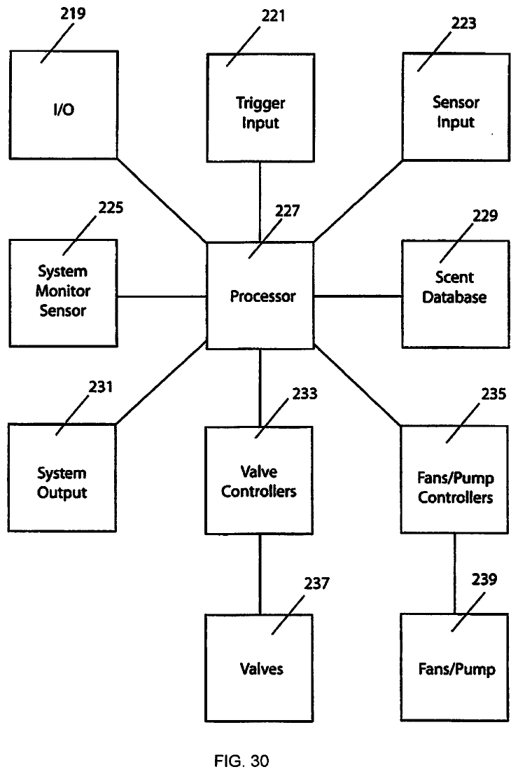

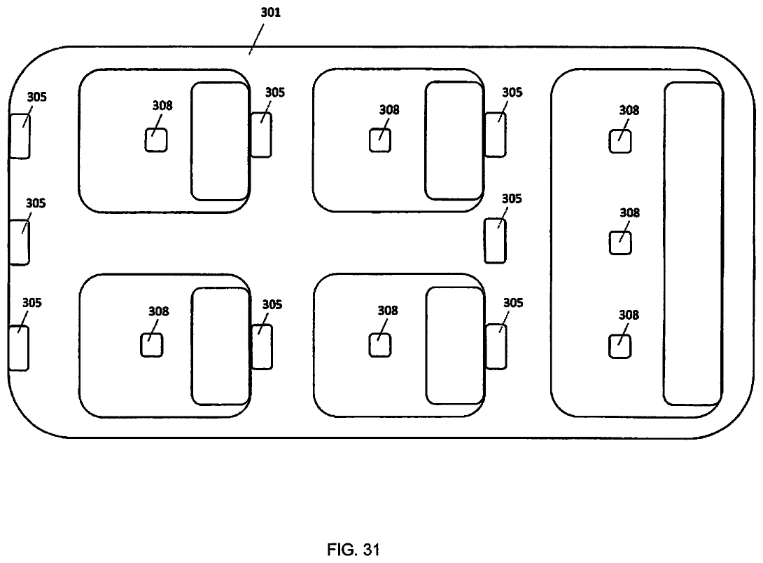

FIG. 30 illustrates a block diagram of possible components of a digital aroma system which can include: an I/O 219, a trigger input 221, a sensor input 223, system monitor sensors 225, processor 227, a scent database 229, a system monitor sensor 225, a processor 227, a scent database 229, a system output 231, valve controllers 233, vales 237, fan/pump controllers 239 and fans/pumps 239. The I/O 219 can be a transceiver that allows communications between the digital aroma system and other media devices, servers, smartphones, servers, other digital aroma system and other computing devices. In an embodiment, the I/O 219 can provide system communications wirelessly through Blue Tooth, Wi-Fi, RFID or similar technologies with other devices, which can provide control signals for releasing fragrances. The trigger input 221 is an input for control signals from nausea input devices such as controllers, user interfaces, etc. In an embodiment, the trigger input 221 can provide system communications wirelessly through Blue Tooth, Wi-Fi, RFID or similar technologies with other devices, which can provide control signals for releasing fragrances.

When the digital aroma system is used, it can go through a startup procedure, which identifies each fragrance cartridge stored in the system. As discussed, the fragrance cartridges can have an identification system, which are read by the system monitor sensors 225. For example, in an embodiment each of the plurality of fragrance cartridges includes an RFID tag that identifies a scent of the dry fragrance cartridge and an RFID reader reads the RFID tags of the fragrance cartridges. The RFID readers can be system monitor sensors 225. The digital aroma system includes a visual display, which can be a system output 231 for displaying the scent of the dry fragrance cartridge. The system can then match the different fragrance cartridges to the various fragrance triggers and store this information in the scent database 229. The system can emit the target fragrance when the corresponding trigger is detected by the trigger input 221 or other signals are detected by one of the sensor inputs 223.

In an embodiment, the digital aroma system can disperse different fragrances in different ways because with each different scent, there is a specific way in which the scent interacts with the air and within the olfactory senses in a human. An example of this difference in scents can be illustrated by comparing citrus and tobacco type fragrances. Citrus type scent molecules travels faster than a tobacco type of fragrance through air.

Graham's Law can be used to compare the effusion rates of different odorous molecules such as lemon and tobacco. Graham's law states that the rate of diffusion or of effusion of a gas is inversely proportional to the square root of its molecular weight. Under ideal conditions, you would smell lemon first because it is composed of ten carbon atoms, eighteen hydrogen atoms, and a single oxygen atom, which gives it a molar mass of 154.25 grams per mole. (A mole is a unit equivalent to about 6*10\hat{ }23 individual molecules). Tobacco, one the other hand, has nine carbon atoms, nine hydrogen atoms, and one nitrogen atom, which adds up to a molar mass of 131.17 grams per mole. Graham's Law says that the rate of lemon effusion divided by the rate of tobacco effusion will equal the square root of the molar mass of tobacco divided by the molar mass of lemon. Thus, users of the digital fragrance system would smell a lemon scent before a tobacco scent due to the higher mass of the lemon scent molecules. In order to compensate for this scent detection difference, the digital fragrance system can adjust the flow rate of the air through the fragrance cartridges based upon the molecular weight. In an embodiment, the airflow rate through the fragrance cartridges can be inversely proportional to the molecular weights of the fragrance molecules. For example, in order to increase the speed of detection of lower molecular weight fragrances, a higher airflow rate can be used.

Citrus type scent molecules have accelerated scent timing within the human olfactory system. The human sense of smell, or olfaction, is a form of chemoreception, which means human noses transduce chemical signals into neural impulses. Human noses possess nearly four hundred olfactory receptors, and each of these bind with a specific molecular feature. Odorous molecules possess multiple features and will trigger different receptors to varying degrees. These stimuli are then transduced into electrical signals that the human brain can interpret the olfactory receptor signals. A lemon scent will trigger receptors that will get the olfactory receptor signals to brain faster as well than lower molecular weight scents.

In an embodiment, the duration of the airflow through the fragrance cartridges can be variable and based upon the strength or perceived strength of the fragrance. A fragrance that has a lower strength may require more airflow through the fragrance cartridge may require more airflow than a higher strength fragrance. In an embodiment, the fragrance system can determine and store the strength values for different fragrances. The fragrance system can be configured to adjust the duration of the airflow through the fragrance cartridges based upon the strength or perceived strength of the fragrance, with a longer duration airflows for weaker strength fragrances and shorter duration airflows for stronger strength fragrances. The fragrance strength can be determined experimentally or based upon measurable chemical characteristics of the fragrance molecules.

In an embodiment, the digital aroma system can include software algorithms that recognize the type of scent that is in the fragrance cartridge 101 based upon identification data on the RFID tag 241 read by the RFID reader 243. By knowing the molecular weight of the fragrance in the fragrance cartridge 101 the digital aroma dispersion system can deploy the proper right number of dry fragrance molecules to into the space required with the proper airflow. Applying identical air pump flow rates and durations to all fragrance cartridges can result in non-uniform fragrance delivery perception of the fragrance recipients. To create a uniform fragrance perception, the inventive digital aroma system can apply variable airflow controls based upon the fragrance being dispersed. The digital aroma system can use a lower airflow rate and can use shorter dispersion durations for higher molecular weight fragrances.

In an embodiment, an airflow rate and duration of airflow can be configured for each different fragrance cartridge so that the fragrances are uniformly sensed by system users. Imperial testing or dry fragrance analysis can determine these airflow rate and duration of airflow settings. Once the airflow rate and duration of airflow are determined, this information can be stored in a memory of the digital aroma system. When the fragrance cartridge is inserted into the digital aroma system, the system can recognize the fragrance from identification information such as an RFID tag and then apply the stored airflow rate and duration of airflow when the fragrance is requested.

In other embodiments, the fragrance cartridges can be configured for the number of dry fragrance particles emitted by the fragrance cartridges can be proportional to the number of dry fragrance beads in the fragrance cartridges since each fragrance bead can provide a uniform surface area. In an embodiment, the number of fragrance beads in the fragrance cartridge can be proportional to the molecular weight of the dry fragrance particle so that the perceived fragrance intensity will be uniform for all fragrance cartridges based upon the same uniform air flow and duration processing. A higher molecular weight fragrance can be recognized by the digital aroma system and when this fragrance is requested, the air flow rate and/or duration through the fragrance cartridge 101 can be lower than that of a lower molecular weight fragrance.

In an embodiment, the digital aroma system can be used with various spaces. The inventive In an embodiment, the system can recognizes the type of fragrance that is in the fragrance cartridge 101 based on the RFID tag 241 and adjusts the air flow speed and the intermittent adjustments for low, medium and high intensity of the fragrance desired in the space. A fragrance that has a lower molecular weight such as citrus can require more air flow to properly disperse the dry fragrance in the space than a higher molecular weight fragrance such as tobacco. If the space is large, the airflow speed and the duration of the fragrance dispersion can be increased. In contrast, for a smaller space, the airflow speed and the duration of the fragrance dispersion can be decreased.

The sensor input 223 can be a sensor that detects ambient signals such as a microphone that detects audio signal or a camera that can detect a video image. The system monitor sensor 225 can be coupled to the digital aroma system components and detect the operation of the components. The scent database 229 can include a list of fragrances information, which can be used to match the fragrance based upon a fragrance identification code signal and then the identification with the valves 237 that must be open to actuate the release of the identified fragrance. The system output 231 can be a visual output, which can be used to inform the system user of system errors or cartridge replacement needs. The valve controllers 233 allow the processor 227 to control the operation of the valves 237. The fans/pumps controllers 235 can be used to allow the processor 227 to control the operation of the fans/pumps. The described digital aroma system components can operate in conjunction to perform various functional actions that can be performed with software running on the processor 227.

In some embodiments, the digital aroma system can recognize video encoded fragrance markers in the video media. The encoded fragrance markers can identify a specific fragrance that is read by the video object recognition system resulting in the identified fragrance being delivered to the user. This feature can be useful in providing a smell before an image corresponding to the fragrance is displayed. For example, the camera point of view in a video may be approaching a fire. The smoke from the fire may be blowing towards the camera and a person at the camera position may smell the smoke before seeing the fire. In order to accurately recreate this scenario the video media may use an encoded fragrance marker for smoke, which is detected by the video object recognition system. The video object recognition system can then emit the smoke fragrance before the fire is shown on the video.

For example, a digital media can include aroma output signals, which can be a video encoded fragrance marker, and the media player can transmit the scent output signal(s) to the trigger input 221 which can be received by the processor 227. The aroma output signals can include aroma identification and the processor 227 can access the scent database 229 to identify the location of the corresponding fragrance cartridge and the valves that must be open to access the identified fragrance cartridge. The processor 227 can then transmit control signals to the valve controllers 233 which actuate the valves 237 to open an airflow path to the identified fragrance cartridge.

In an embodiment the trigger input can be transmitted within a short-range proximity through a device such as a Bluetooth receiver or other local communications device. The aroma system can be used with a mobile device such as a smart phone that is carried by the user. When the user walks within a museum to different exhibits, the trigger input 221 of the digital aroma system can detect trigger signals from different exhibits as the user walks and the aroma system can emit the scent as commanded by the detected trigger signals. In other embodiments, the present invention can be used in many different individual educational settings like museums to provide a cost effective sensory experience using media, software, maintenance and aroma.

In an embodiment, the sensor input 223 can be a camera and the processor 227 can run a video object recognition software that receive video signals from the sensor input 223 camera and recognize objects and/or environments which may induce nausea such as winding roads or heavy traffic. In an embodiment there may be a known time delay between the actuation of the digital aroma system to output a target fragrance and the user smelling the fragrance. The video object recognition system can identify the fragrance video object and/or environment trigger and identify the fragrance that is associated with the trigger. The digital aroma system can then actuate the trigger associated fragrance delivery before the trigger object or environment is displayed by the known time delay period so that the fragrance is delivered to the viewer at the moment when the trigger object or environment is being displayed.

In an embodiment the digital aroma system can use a microphone as a sensor input 223 that can be triggered the correct aroma with sound recognition software running on the processor 227 that recognizes audio commands and disperses the correct aroma based on the audio commands. The audio recognition system can receive the audio signals and use the scent database 229 to identify the fragrance associated with the audio signals. The processor 227 running audio recognition software can then control the valves 237 and fans/pumps 239 to actuate the fragrance delivery.

In an embodiment, the digital aroma system can include software running on the local processor that can communicate through the I/O 219 to the Internet to a cloud service. This communication capability can be used with the system monitor sensor 225 for remote monitoring of the cassettes and fragrance cartridges, the duration of the number of uses, and remotely monitors the health of the pump and/or fan and health in the digital aroma system to ensure the system components are working properly. If errors or end of life are detected in any of the system components, the processor 227 of the digital aroma system sends alerts to a user or system administrator identifying the errors through the system output 231 when something is not working properly. The system output 231 can be a visual display, an audio output device and/or a digital wireless communication output.

In another embodiment, the digital aroma system can be used with a fragrance sensor that can measure the intensity or concentration of the dry fragrance particles in the air space around the digital aroma system. With reference to FIG. 30, the sensor input 223 can be a fragrance sensor(s). By detecting the concentration of the fragrance, the system can be configured to maintain a fragrance concentration within a specific range. With reference to FIG. 31, the fragrance concentration sensors 308 can detect the fragrance concentration within a vehicle 301 and the fragrance system can be configured to emit the dry fragrance from fragrance emission unit vents 305 when the system receives a manual nausea input or predicts passenger nausea based upon rotation or acceleration and duration above predetermined threshold values.

Different vehicles can have different passenger accelerations when moving at the same velocity on the same road. For example, passengers in a bus or a van can be much higher than the passenger positions in a low riding sports car. The bus can have a soft suspension which causes the vehicle to rotate in roll as the bus travels around a turn. In order to accurately measure the passenger movements, the movement sensors can be mounted in the vehicle at the same or similar position as the passengers.

While the patent application describes the use of the inventive anti-nausea system as vehicles such as cars, buses, and vans which have wheels that travel over roads. However, this sensor system based nausea prediction system can used with any other type of non-road traveling vehicle such as trains, helicopters, airplanes, hovercrafts, hydrofoils, boats, ferries, etc. When the vehicle routes are known the system can predict possible nausea locations based upon a database of routes and known nausea locations. The system may also take into account weather conditions. For example, boats and airplanes will experience additional movements when these vehicles travel through storms and rough weather. In an embodiment, the system may obtain current and predicted weather and use this information to predict possible nausea locations and emit the anti-nausea fragrances to passengers as described.

The fragrance system can be configured to direct anti-nausea fragrance to specific passenger seats or equally (or unequally) distribute the anti-nausea fragrance to all vehicle passenger seats. In an embodiment, the system can detect the number and locations of the passengers with seat sensors which can detect the weight of the passengers on the seats. Based upon this information, the system can only direct the fragrances to the vehicle passengers to conserver the fragrances. For many trips, the only passenger is the driver and the system can only direct the fragrance towards the driver to conserve the fragrances. It is well known that people sitting in the back of the vehicle are more prone to getting motion sickness. In an embodiment, the system can asymmetrically distribute the anti-nausea fragrances based upon the seating positions with more fragrances being distributed to the back seats.

In an embodiment, the system can detect the concentration distribution of the fragrance with a plurality of fragrance sensors 308 placed in a plurality of locations in the vehicle 301 such as above each passenger seat. When multiple sensors 308 are used, the sensor network can determine the fragrance emission pattern based upon the detected fragrance concentrations of the sensors. This fragrance information can be used to properly orient the fragrance output from the system to asymmetrically direct fragrance to the passenger(s) in the vehicle. In an embodiment, the system detect the locations of people into the vehicle 301 with sensors such as proximity sensors 308 which can be located by each seat in the vehicle. When people are not in one or more of the seats, the system can stop the emission of fragrances from the fragrance emission vents 305 to these locations. When a person enters the vehicle 301, the system can detect the person or people and the fragrance emission unit 305 can emit a fragrance, which can be experienced by people in the vehicle 301. For example, the system can use sensors in the seats such as seatbelt weight sensors can be used to identify the seats where passengers are sitting.

In addition to detecting the concentration of the system fragrances, the fragrance sensors 308 can also be used to detect outside odors which can influence the user's nausea. These outside odors can include: exhaust fumes, toxic gases (CO, carbon monoxide), body odor, vomit, flatulent, biomarkers, etc. The sensor system can respond by emitting fragrances and control the ventilation system to increase the air flow through the vehicle when offensive odors are detected.

The fragrance sensor 308 can be based upon sensor mechanisms such as chemo sensors or by gas chromatography, which provides information about volatile organic compounds. Electronic fragrance sensors 308 can include a detection system and a computing system. The detection system can consist of a sensor set, which can contact fragrance particles and react by producing a change of electrical properties. The fragrance sensor can be sensitive to all fragrance molecules but can be able to distinguish different fragrance particles. The fragrance sensor may use sensor arrays that react to volatile compounds on contact: the adsorption of volatile compounds on the sensor surface causes a physical change of the sensor. A specific response is recorded by the electronic interface transforming the signal into a digital value. Recorded data are then computed based on statistical models. In an embodiment, the fragrance sensors can be metal-oxide-semiconductor (MOSFET) devices—a transistor used for amplifying or switching electronic signals. Molecules can enter the fragrance sensor area and will be charged either positively or negatively, which should have a direct effect on the electric field inside the MOSFET. Thus, introducing each additional charged particle will directly affect the transistor in a unique way, producing a change in the MOSFET signal that can then be interpreted by pattern recognition computer systems.

In the present digital aroma system invention, the user can easily change the fragrance cartridges and may only need to replace the cartridges every few months depending upon the scent use. In an embodiment, the digital aroma system can monitor the number of times each of the fragrance cartridges is used. When the life of the cartridge is reaching its end, the system can warn the user that the cartridge needs to be replaced. Thus, the cartridge only that needs to be replaced as needed. The longevity of each dry fragrance infused beaded cartridge is anywhere from 1,000-4,500 dispersions. In other embodiments, fragrance cartridges with larger chambers that hold more fragrance infused substrate materials can last longer and provide additional fragrance dispersions.

The present digital aroma system invention also addresses the issue of ease of replacement of the fragrance cartridges by the consumer. The digital aroma system allows the swapping out of several fragrances simultaneously by removing and replacing a single cassette of the digital aroma system. The cassette can contain six or more individual fragrance cartridges containing dry fragrance infused substrate materials. In other embodiments, the cassette is not limited to six fragrance cartridges. For example, the cassette can hold a single fragrance cartridge and in other embodiments the cassette can have couplings to hold ten to twenty or more fragrance cartridges and in a cassette system. In addition the consumer can also change each individual aroma cartridge within the cassette system be simply exchanging each aroma cartridge within the cassette or replacing the entire cassette.

The digital aroma system can include a cassette having a manifold, which holds a plurality of fragrance cartridges. The manifold has air inlets and scent outlets that are coupled to the fragrance cartridges which can have hollow housings which are filled with dry fragrance infused particles such as balls or other loose objects. The cartridge housings can have couplings such as threads or tabs, which can provide a gas tight connection between the cartridges and the manifold. The couplings also allow users to replace or change the fragrance cartridges. The cartridges can also have identification mechanisms, which provide an identification signal output such as a radio frequency identification tag. The identification signal output can identify the fragrance in the cartridge and control the number of fragrance outputs that the cartridge can provide. The digital aroma system can have readers, which can read the identities of the fragrance in the cartridges and store this fragrance and cartridge location information so that desired fragrance can be controlled to emit by the digital aroma system.

In different embodiments, the described fragrance aroma dispersion system can be controlled and monitored by various different computer interfaces. For example, in an embodiment, an automotive interior can have integrated hardware components that emit multiple selected scents into the car's interior. The driver or a passenger can select fragrances, which are automatically infused into the air system, which emits the scent throughout the car's interior. In an embodiment, up to 5 selectable fragrances including an anti-nausea fragrance to personalize the driving experience. In other embodiments, any number of fragrances or combinations of fragrances can be selected. The scent cartridges provide ease of use for replacement by the car dealer or by the consumer.

In an embodiment, the fragrance system can be a component of a connected control platform, which can include one or more digital aroma dispersion systems in communication with a system server that can monitor the operation of the systems. By monitoring the digital aroma dispersion systems, the control platform can perform intelligent inventory control for efficient fragrance cartridge efficiency. In an embodiment, the control platform monitors system usage by receiving fragrance cartridge usage information for each of the digital aroma dispersion systems. The system server can collect the operation and system usage data. By knowing the rated number of dispersions for each fragrance cartridge, the server can provide alerts to the individual system users for fragrance cartridges to replace individual cartridges. The warning messages can be transmitted to a mobile smart phone or a display on a vehicle.

In some embodiments, the server monitoring system may even make suggestions for improving the efficiency of any of the installed systems. For example, a first fragrance cartridge may be used at an average rate of 10 dispersions per day and a second fragrance cartridge may be used at an average rate of 5 dispersions per day in a single system by a specific user the total dispersion rating is 3,000, then the server can predict that the first fragrance cartridge will last 300 days and the second fragrance cartridge may last 600 days. The server can transmit electronic warning signals to the system user when the fragrance cartridge has approximately 1 month or 30 days of remaining dispersions. Because these warnings are time based, they are not indicative of a specific percentage dispersions remaining.

It can be inconvenient to replace single fragrance cartridges in a multi-cartridge system. In an embodiment, the server can determine that the user has a favorite fragrance that is used more often than the other fragrances based upon historical data and recommend to the user that multiple cartridges of the favorite fragrance be placed in the system. The system can then alternate dispersions between the two identical fragrance cartridges so that multiple fragrance cartridges can be depleted at the same or a similar rate and when fully depleted, the multiple fragrance cartridges can be replaced at the same time. Similarly, based upon infrequent use, the system may recommend using a lower capacity fragrance cartridge for less popular fragrances that has a history of lower rates of dispersion over time than the more popular fragrances, so multiple fragrance cartridges will needing replacement at the same time.

In an embodiment, the fragrance dispersion module can be installed in a vehicle with multiple fragrance cartridges. The fragrance dispersion module can receive control systems and transmit fragrance cartridge information to a smart phone and/or an in-dash mobile control unit. A driver or passenger of the vehicle can interact with the smart phone and/or an in-dash mobile control unit to control the operation of the fragrance dispersion module. The smart phone and/or an in-dash mobile control unit can communicate with other computing devices that are remote from the vehicle such as servers that receive information from many different fragrance dispersion modules, personal computers and mobile computing devices operated by the vehicle drivers or passengers, and other computing devices. These system components can share information so that the system functions optimally.

The system servers can also use the cumulative data measurements to collect data for a large number of installed fragrance dispersion systems. This data can be grouped by region, user information, season, temperature, country or any other distinguishing characteristic. By understanding the preferences of a large user group, the system servers can provide business intelligence for advanced planning, real time trending, geographic trending and dashboard reporting. For example, each fragrance dispersion system can transmit fragrance dispersion information to a central server(s) and this data can predict the rate of consumption for each type of fragrance cartridge. This information can then be used to order additional fragrance cartridges so that adequate quantities will be available when needed. This information can also be used to identify popular and unpopular fragrances. If a fragrance is not being consumed in reasonable quantities, the system may determine that a specific unpopular fragrance may have a quality control problem and the inventory may need to be replaced. Alternatively, the system may request that an unpopular fragrance be discontinued or modified to be more accepted by consumers. This information may also be used to determine fragrance trends so that new fragrances can be developed that are similar to the most popular existing fragrances.

The data can also be used by the server to identify correlations between user demographics and preferred fragrances. For example, the system may detect differences in fragrance preferences between men and women and children where men prefer an outdoors forest fragrance, women prefer a floral fragrance and children may prefer a beach fragrance. By knowing the fragrance preferences based upon user demographics, the server can make fragrance preference predictions for future consumers based upon specific user, geography, vehicle type, season, etc. If an automobile buyer decides to purchase a vehicle with the fragrance dispersion, the user information can be provided and used to predict a set of fragrance cartridges that are most likely to be popular with the consumers. Alternatively, the consumer may be able to select the individual fragrances to be supplied with the vehicle.

The fragrance consumption information from the data can be displayed in various ways on system servers including: numerical data, graphical data showing fragrance consumption trend lines in a vertical axis over time on a horizontal axis, bar or pie charts, current fragrance levels for a specific fragrance system, etc. In an embodiment, the fragrance consumption information can be transmitted to the integrated mobile control and/or smart phone so that the users will know their consumption rates, fragrance levels, fragrance cartridge refills needs and compare their personal use information to general user information.

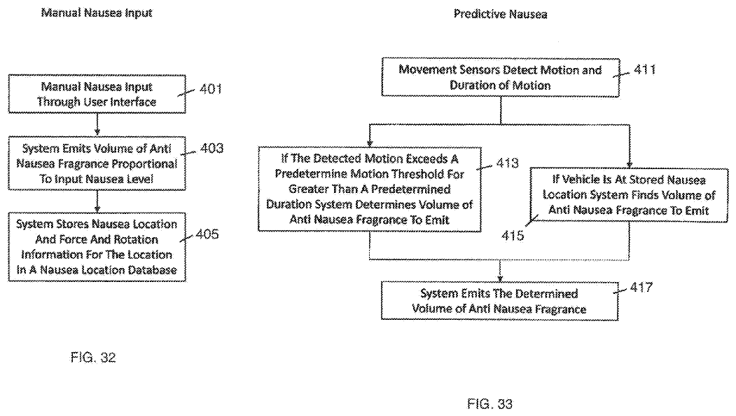

In an embodiment, the inventive system can perform various processes to detect and predict vehicle movements that will result in nausea. With reference to FIG. 32, a flowchart is illustrated that describes a manual method for actuating the anti-nausea fragrance output and recording. As discussed, when the user become nauseas when traveling in a vehicle, the user can press a button on a user interface to inform the system of the nausea condition 401. The system can respond by emitting the anti-nausea fragrance in a volume that is proportional to the nausea level input by the user through the user interface 403. In addition to emitting the anti-nausea fragrance, the system can also record the nausea information which can include: the location, the forces and movement of the vehicle prior to the user becoming nauseous. The system can also identify the rotation and forces that were detected by the movement sensors prior to the user's nausea input and this information can be stored by the system in a nausea location database 405.

This information can be stored in a database for the individual users. Different people have different nausea susceptibilities. Some people get motion sick very easily while other people almost never suffer from motion sickness. Some people can be more sensitive than other people to certain odors and certain types of motions. In an embodiment, the system can cumulatively group the users into different categories of nausea susceptibilities. For example, as nausea data is collected, the system can identify users who frequently get nauseous, occasionally get nauseous, periodically get nauseous, rarely get nauseous and almost never get nauseous. These groups can be determined based upon nausea inputs per time. A passenger who gets nauseous several times per week or month will be in a more nausea susceptible group than a passenger who get motion sick once or twice a year. The system can compare the user's nausea susceptibility based upon the magnitudes of motion and forces and durations detected prior to each user inputting a nausea level through the user interface. A user who can handle a higher rate of motion and forces and duration will be placed in a less nausea susceptible group than a person who gets nauseous with the same rate of motion and forces and duration. Each of these groups can have specific ranges of motions and road locations that are likely to result in nausea. The system users can each be given a nausea susceptibility rating and the nausea locations and intensity data can be shared with all system users.

As the system is used, the users will travel in vehicles and input road locations where nausea occurs. The system can receive the nausea locations and nausea intensities and this information can be stored in a database. The database can be used to create road nausea maps which can identify the detected road nausea areas. The different groups of users will have different nausea locations with more sensitive people having many more nausea locations than less motion sickness sensitive people. Thus, a nausea map for a more sensitive group will have more predicted nausea locations than the nausea map for a less sensitive group of system users. The nausea maps can be shared with other system users who are in the same sensitivity group.

There will be overlap of nausea conditions with the different groups. For example, the conditions and locations that cause users who almost never get nauseous to be nauseous will be applicable to all other groups. The conditions and locations that cause users who rarely get nauseous to be nauseous will be applicable to all more susceptible groups. In an embodiment, the system can identify the passengers in a vehicle and the nausea susceptibility levels for each of the passengers based upon the stored nausea history. This nausea group database information can be used by the system for future trips. When the vehicle or user with a portable fragrance system travels, the system can look up the nausea map that covers the vehicle's location for the group of passenger(s) in the vehicle. The system can identify the locations had been a nausea location for the user and other users in the same nausea group. Based upon this information, the system may emit an anti-nausea fragrance as the vehicle travels over a road that is likely nausea inducing road location based upon prior user nausea data or detected forces applied to the passengers including: rotation, acceleration, and duration that exceed predetermined threshold values for the nausea group.

In an embodiment, the system can store a nausea value for all nausea locations on a map and adjust the volume of the anti-nausea fragrance based upon the nausea value and the nausea susceptibility of the vehicle passengers. For example, the system can have nausea values between 1 and 100. These nausea values can be broken up into 5 groups: level 1 can be 1-20, level 2 can be 21-40, level 3 can be 41-60, level 4 can be 61-80 and level 5 can be 81-100+. The 5 different levels can correspond to different volumes and/or durations of the anti-nausea fragrance. In a linear example, a level 1 nausea location can cause the vehicle to emit 1 second duration of the anti-nausea fragrance, a level 2 nausea location can cause the vehicle to emit 2 second duration, etc.

The volumes and or durations of the anti-nausea fragrance can also be adjusted based upon the nausea susceptibility of the vehicle passengers. In an embodiment, the volumes and/or durations of the anti-nausea fragrance can be based upon most nausea susceptible passengers. In order to prevent or minimize the emission of the anti-nausea fragrance, the system can adjust the fragrance emissions when lower nausea susceptibility passengers are in the vehicle. In an embodiment, the system can categorize all passengers into different nausea susceptibility classes. A class 1 passenger may easy be nauseas while a class 5 passenger may very rarely be nauseas due to road conditions. The system may divide the duration or volume of the anti-nausea fragrance output by the class level of the passengers. If a vehicle has at least one class 1 passenger, the full volume or duration of the anti-nausea fragrance can be output by the system. However, if the most nausea susceptible passenger is a class 3 passenger, the volume or duration of the anti-nausea fragrance can be divided by 3. if the most nausea susceptible passenger is a class 5 passenger, the volume or duration of the anti-nausea fragrance can be divided by 5. In an embodiment, testing can be performed and based upon the feedback of the passengers, the system can be properly adjusted to emit a sufficient volume of the anti-nausea fragrance for all varieties of passengers.

While passengers may normally have the same nausea group association over time. However, there can be situations where the passenger has an increased susceptibility to motion sickness. For example, the passenger may have a medical condition which temporarily alters the nausea susceptibility. These conditions can include: illness, hang over, headache, etc. In an embodiment, the system can allow users to make manual adjustments to their associated nausea group. If a user knows that he or she is feeling more susceptible to nausea, the user interface can be actuated to temporarily adjust the user's associated nausea group.

A specific section of a road can have different nausea probabilities based upon different traffic conditions. In heavy traffic the vehicles may be traveling in a start/stop manner with increased concentration of exhaust fumes which can increase the likelihood of nausea. As traffic decreases, the vehicles can assume a more steady velocity and the exhaust fume concentration can decrease and the likelihood of nausea can decrease. In light traffic, the velocity of the vehicles can increase and the likelihood of nausea can decrease for straight roads. However, as the vehicle speed increases on windy roads, the centripetal forces applied to the vehicle passengers can increase which can result in increase the likelihood of nausea. In an embodiment, traffic speed can be detected based upon existing real time traffic maps and databases.

This movement data can be used to identify and predict future conditions that can result in nausea. With reference to FIG. 33, a flowchart showing a predict nausea process is illustrated. The system can detect the rotation and acceleration movements of the vehicle and user and the duration of the motions in real time 411. The system can identify rotations and forces that are equal to or greater than the rotations and forces that resulted in nausea. If the detected motion exceeds a predetermined motion threshold for greater than a predetermined duration, the system can calculate and determine a volume of anti nausea fragrance to emit 413. The system can also identify the nausea locations that were stored in the database by the user and/or other system users. If the detected movement or the prior nausea location are detected with the matching vehicle speed, the system can also determine the volume of anti nausea fragrance to emit 415. The system can then output the anti-nausea fragrance in a volume that corresponds to the predicted nausea level 417.

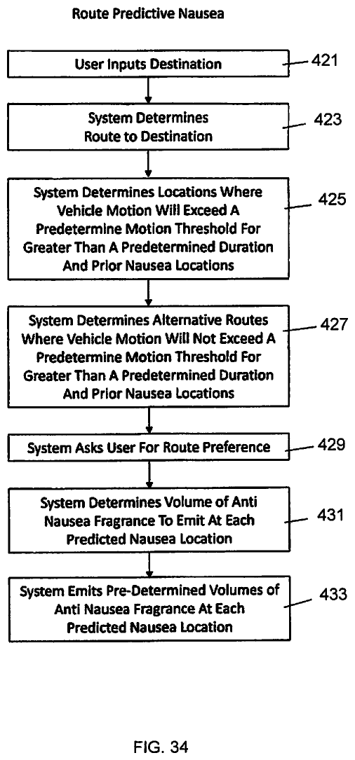

Many people use GPS devices to determine driving routes to a destination. In an embodiment, the fragrance system can analyze the route and determine if there are any known or predicted nausea locations. In order to detect the rotation, the system can simply identify the curves and normal vehicle speeds on the route. The centripetal forces can be calculated by dividing the square of the velocity by the radius of the road curvatures, F_{centripetal}=V²/r. Road curves are frequently in groups which can increase the likely hood of nausea. The system can calculate the potentially problematic roads by determining that the centripetal force above the threshold level is repeated for at least a specific duration of time. With reference to FIG. 34, the inventive system can be used to provide route predictive nausea services. The user can input a destination 421. The GPS system can respond by determining a route to the destination 423. The system determines locations on the route where the vehicle motion will exceed a predetermine motion threshold for greater than a predetermined duration and the system also determine prior nausea locations for the specific user or uses who have similar nausea reactions 425.

The system can then analyze possible routes and identify the locations that may result in nausea based upon the passengers' nausea group, known nausea location, and predicted vehicle motions based upon the passenger nausea susceptibility, road speed and curvatures of the route. The system can list possible routes with information and likelihood of nausea. The system may suggest a route which may be longer but less likely to result in motion sickness if a route that is less likely to result in motion sick if available. The system can issue an option to select the less nauseous route and the user can select the desired route 429. The system can also inform the user of the predicted time when the desired route has the optimum traffic level to minimize the likelihood of nausea for the passenger nausea group. The system can then determine the volumes of the anti-nausea fragrances 431. As the vehicle travels through the known route, the system can emit the predetermined volumes of the anti-nausea fragrances at the locations of predicted nausea or actual nausea 433.

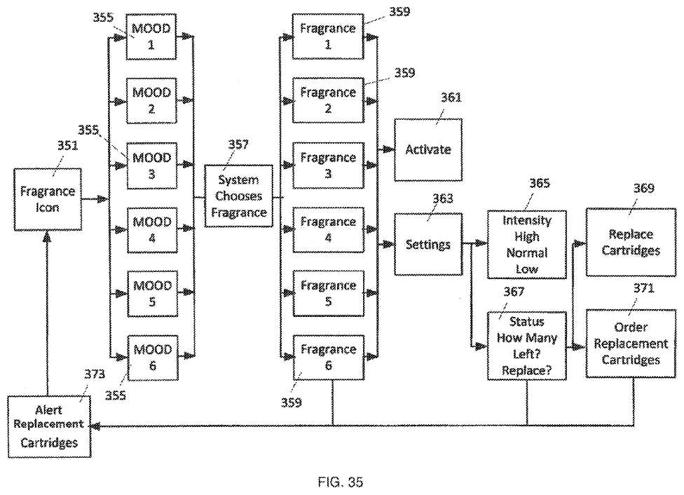

With reference to FIG. 35 the fragrance systems can be part of an in-vehicle system used to create a mood scent experience that can coordinate lighting, scent and audio systems which are all integrated. Each of these experiences can be coordinated by a desired mood or activity. For example, a system may have a user interface with a plurality of moods and/or activities that can be selected by a system user such as: dinner party, Friday night, festival, workout, road trip, studying, game day, BBQ, family time, wake up, focused, raining day, in love, wind down, memory lane and any other mood/activity. When a mood/activity is selected by pressing a button 351, voice control or any other input, the system can cause the lighting, scent and music to produce outputs that are coordinated with the selected mood/activity. The lighting output can be blue for increased productivity, positive environment, reduce depression, less anxiety and peacefulness. The lighting output can be yellow for increased competence, happiness, light and joyfulness. The lighting output can be red for increased excitement, love, enthusiasm and passion. The lighting output can be green for increased rejuvenation, freshness and vigor.

The fragrance system used with the sensory experience network system can be used for specific purposes. As discussed, when the system predicts or received a manual input of motion sickness, the system can emit the anti-nausea fragrance and provide audio output and lighting which can each help to mitigate the nausea. The user interface of the integrated mobile control and/or smart phone may have “nausea” input buttons that can result in the emission of the anti-nausea fragrance that can reduce nausea for a driver or passenger who is not feeling well or is carsick. For example, fragrances like peppermint, ginger, lavender, chamomile, cardamom, coriander, fennel, nutmeg, aniseed, star anise, bergamot, lemon, spearmint, grapefruit and geranium can help reduce nausea. If the nausea symptoms persist or escalate, the driver can be instructed to slow down or take an alternative route which has fewer curves. In extreme situations such as a discomfort level 4 or 5, the driver can be instructed to pull off of the road and stop. Similarly, if the vehicle is an autonomously driven vehicle, the vehicle control system can be instructed to slow down, take an alternative route which has fewer curves or pull off of the road to stop in extreme situations.

Studies have been performed on the effects of different fragrances on nausea. The Iranian Red Crescent Medical Journal, published a 2012 study of the Effect of Mint Oil on Nausea and Vomiting During Pregnancy. In the study, the severity of nausea showed a decreasing trend (especially in 4th night) in the mint test group and increased in the control group. Pasha, H., Behmanesh, F., Mohsenzadeh, F., Hajahmadi, M., & Moghadamnia, A. A. (2012). Study of the Effect of Mint Oil on Nausea and Vomiting During Pregnancy. Iranian Red Crescent Medical Journal, 14(11), 727-730. http://doi.org/10.5812/ircmj.3477 Integrative Medicine Insights published an article The Effectiveness of Ginger in the Prevention of Nausea and Vomiting during Pregnancy and Chemotherapy. The paper concludes that the best available evidence demonstrates that ginger is an effective and inexpensive treatment for nausea and vomiting and is safe. Lete, I., & Allué, J. (2016). The Effectiveness of Ginger in the Prevention of Nausea and Vomiting during Pregnancy and Chemotherapy. Integrative Medicine Insights, 11, 11-17. http://doi.org/10.4137/IMI.S36273 Phytomedicine published a paper A multi-center, double-blind, randomised study of the Lavender oil preparation Silexan in comparison to Lorazepam for generalized anxiety disorder. The paper states that lavender oil preparation silexan appears to be an effective and well tolerated alternative to benzodiazepines for amelioration of generalised anxiety. Woelk, H., & Schläfke, S. (2010). A multi-center, double-blind, randomised study of the Lavender oil preparation Silexan in comparison to Lorazepam for generalized anxiety disorder. Phytomedicine, 17(2), 94-99. The Iranian Red Crescent Medical Journal published a 2014 paper on the effect of lemon inhalation aromatherapy on nausea and vomiting of pregnancy. The paper states that inhalation aromatherapy with Lemon essential oil showed that this method could reduce nausea and vomiting during pregnancy (NVP). In contrast to chemical medications, aromatherapy has useful effects on physical and psychological health and might be useful as an alternative approach in the treatment of NVP. In an embodiment, the anti-nausea fragrance is a proprietary blend of dry of at least 2 of the following scents: Peppermint, Spearmint, Lemon, Ginger, and Lavender. The percentages of each scent of the proprietary blend varies on the combination and does not always include all of these ingredients.

The fragrance system can also be used to produce other specific purpose sensory experiences. For example, a cinnamon fragrance can be used for concentration and focus. Researchers from Wheeling Jesuit University found that those who smelled a cinnamon fragrance improved in cognitive functions like visual-motor response, working memory and attention span. A pine fragrance can be used to decrease anxiety, according to a Japanese study in which participants reported significantly lower depression and stress levels. The research also discovered that anxious subjects had a greater feeling of relaxation after indulging in the pine fragrance. Fresh-cut grass fragrances can make users more joyful. In a study (ISOT/JASTS 2004), researchers found that taking a vanilla bean fragrance elevated participants' feelings of joy and relaxation. A small study out of Wheeling Jesuit University found that smelling a peppermint fragrance could be linked to greater cognitive stamina, motivation and overall performance. A 2010 study, Nat. Prod. Commun. 2010 January; 5(1):157-62, “Stimulating effect of aromatherapy massage with jasmine oil” found that not only does the smell of jasmine create a sense of alertness, it can also serve as a way to help with depressive thoughts. Researchers found that the stimulating effect of jasmine fragrance can aid in the relief of depression and can lead to an uplifted mood. Research has suggested that the smell of apple fragrance may actually help ease a migraine. One 2008 study showed that those who found the scent appealing had a noticeable reduction in headache symptoms as well as shortened migraine episodes. Previous studies on a green apple fragrances have also found the scent may help control feelings of anxiety during stressful moments. In some embodiments, vehicle model specific or vehicle make specific fragrances can be developed and these special fragrances can be supplied with the vehicle model or all vehicles produced by a vehicle make.

The sensory experience network system can also coordinate audio signals within the vehicle with the lighting and scent. Music appropriate for each of the moods and activities can be played in a random sequence of songs or ambient background sounds. In some embodiments, the integrated mobile control and/or smart phone may have special fragrances for assisting with special physical conditions such as alertness and nausea. In an embodiment, the user interface of the integrated mobile control and/or smart phone may have an “alert” button that can result in a fragrance that can improve the alertness of the driver or passengers. For example, fragrances like lemon, orange, cinnamon, mint and rosemary can help boost energy and alertness.

Table 1 provides a listing of light colors and the physical reaction that can be improved by the light colors.

TABLE 1

| Color | Reaction |
|---|---|
| Blue | Anti-Nausea, Increase Productivity, Positive environment, |
| | Reduce Depression, Less Anxiety, Peaceful |
| Yellow | Mellow, Competence, Happiness, Light, and Joyful |
| Red | Excitement, Love, Enthusiasm, and Passionate |
| Green | Rejuvenating, Freshness, and Vigor |

Table 2 provides a listing of fragrances and the physical reaction that can be improved by the fragrances.

TABLE 2

| Fragrance | Reaction |
|---|---|
| Cinnamon | Concentration, and Focus |
| Ginger | Anti-Nausea |
| Lemon | Anti-Nausea, Concentration, Calming, Clarifying, |
| | Joyful, and Happy |
| Jasmine | Calm Nerves, Uplifting, Confidence, Optimism, and |
| | Revitalized Energy |
| Lavender | Anti-Nausea, Calming Emotional Stress, Soothing Effect |
| | on Nerves, and Relieves Nervous Tension |
| Peppermint | Anti-Nausea, Energy Booster, Invigorates the Mind, |
| | and Stimulates Clear Thinking |
| Rosemary | Pick-Me-Up, Improves Memory Retention, and |
| | Stimulates Mental Activity |

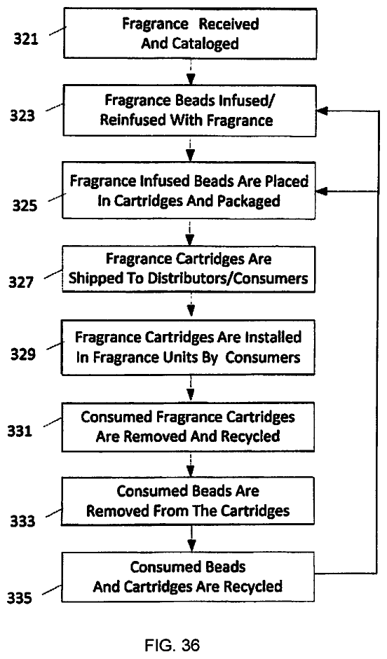

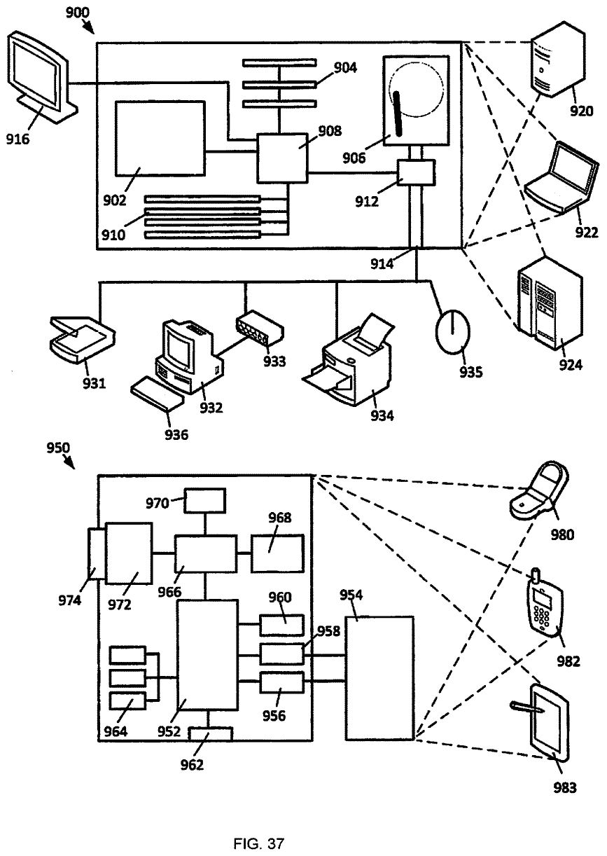

With reference to FIG. 36, a flow chart of an embodiment of a fragrance system is illustrated. In an embodiment, a user can select access the fragrance system controls by pressing a fragrance icon 351 on a user interface of the integrated mobile control and/or smart phone. The system can respond by displaying a plurality of moods or activities 355. The user can then select a desired mood or activity 355. The system can choose a fragrance 357 or the system can be pre-configured with fragrance(s) 359 that correspond to the different moods or activities 355. The system can then actuate and disperse a fragrance or fragrances 359 associated with the user selected mood 355.

The user may need to adjust the system control settings 363 and the system user interface can have adjustments for the dispersion intensity setting 365. In the illustrated example, the user can configure the dispersion output for the fragrance cartridge to low, high or normal 365. In other embodiments, the system can have any other variable settings. The fragrance system can keep track of the activations 361 and dispersions for each fragrance cartridge and determine the status of each fragrance cartridge and calculate the number of dispersions left and if there are more dispersions available for each cartridge 367. The system can determine if the cartridge needs to be replaced 369 before the fragrances are fully depleted from the cartridges. The fragrance system may automatically order replacement fragrance cartridges 371 when the number of remaining fragrance dispersions is low. If the cartridges need to be replaced, the system can provide an alarm which can provide a signal to the user interface that may instruct the user the user to replace the cartridges 373. The system may instruct the user on how to replace the fragrance cartridges when they are depleted. The process can be repeated after each fragrance dispersion.

The described software can be used to control cartridge efficiency, monitor usage, data collection and alerts for replenishment. The network system can be linked to supply chains for consistent usage, automated replenishment and billing based upon predicted and actual usage. System planning based upon real time trending, geographic trending, dashboard reporting. Web interface programming of devices (SDK), smart phone compatible.

With reference to FIG. 36, in an embodiment, a specific process can be performed construct the fragrance cartridges and recycle the fragrance cartridges components. The fragrance oils or other fragrance media are received and cataloged 321. The beads are placed in or near the fragrance oil and the fragrance oils or other fragrance media are infused into beads 323. The bead fragrance infusion process can take up to four weeks. After the beads have been infused with a fragrance, the fragrance infused beads are placed in fragrance cartridges and the fragrance cartridges can be packed 325. The filled fragrance cartridges are shipped to distributors, dealers and/or directly to consumers 327. The fragrance cartridges are then inserted into the fragrance dispersion units and used by the end consumers 329. Air is blown through the fragrance cartridges and the dry fragrances are blown off of the fragrance beads. At the end of the dispersion cycle life, the fragrance cartridges are removed from the fragrance dispersion units and replaced with new fragrance cartridges. The depleted fragrance cartridges can then be recycled 331. In an embodiment, the fragrance cartridges are disassembled and the fragrance depleted beads are removed from the cartridges 333. The beads and cartridges can be recycled 335. The used beads can be cleaned and re-infused with the fragrances 323. Once re-infused, these fragrance beads can be placed in a fragrance cartridge 325 and the consumption process can be repeated.

FIG. 37 shows an example of a generic computer device 900 and a generic mobile computer device 950, which may be used to implement the processes described herein, including the mobile-side and server-side processes for installing a computer program from a mobile device to a computer. Computing device 900 is intended to represent various forms of digital computers, such as laptops, desktops, workstations, personal digital assistants, servers, blade servers, mainframes, and other appropriate computers. Computing device 950 is intended to represent various forms of mobile devices, such as personal digital assistants, cellular telephones, smartphones, and other similar computing devices. The components shown here, their connections and relationships, and their functions, are meant to be exemplary only, and are not meant to limit implementations of the inventions described and/or claimed in this document.

Computing device 900 includes a processor 902, memory 904, a storage device 906, a high-speed interface 908 connecting to memory 904 and high-speed expansion ports 910, and a low speed interface 912 connecting to low speed bus 914 and storage device 906. Each of the components processor 902, memory 904, storage device 906, high-speed interface 908, high-speed expansion ports 910, and low speed interface 912 are interconnected using various busses, and may be mounted on a common motherboard or in other manners as appropriate. The processor 902 can process instructions for execution within the computing device 900, including instructions stored in the memory 904 or on the storage device 906 to display graphical information for a GUI on an external input/output device, such as display 916 coupled to high speed interface 908. In other implementations, multiple processors and/or multiple busses may be used, as appropriate, along with multiple memories and types of memory. Also, multiple computing devices 900 may be connected, with each device providing portions of the necessary operations (e.g., as a server bank, a group of blade servers, or a multi-processor system).

The memory 904 stores information within the computing device 900. In one implementation, the memory 904 is a volatile memory unit or units. In another implementation, the memory 904 is a non-volatile memory unit or units. The memory 904 may also be another form of computer-readable medium, such as a magnetic or optical disk.

The storage device 906 is capable of providing mass storage for the computing device 900. In one implementation, the storage device 906 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier may be a non-transitory computer- or machine-readable storage medium, such as the memory 904, the storage device 906, or memory on processor 902.

The high speed controller 908 manages bandwidth-intensive operations for the computing device 900, while the low speed controller 912 manages lower bandwidth-intensive operations. Such allocation of functions is exemplary only. In one implementation, the high-speed controller 908 is coupled to memory 904, display 916 (e.g., through a graphics processor or accelerator), and to high-speed expansion ports 910, which may accept various expansion cards (not shown). In the implementation, low-speed controller 912 is coupled to storage device 906 and low-speed expansion port 914. The low-speed expansion port 914, which may include various communication ports (e.g., USB, Bluetooth, Ethernet, wireless Ethernet), may be coupled to one or more input/output devices, such as a keyboard 936 in communication with a computer 932, a pointing device 935, a scanner 931, or a networking device 933 such as a switch or router, e.g., through a network adapter.

The computing device 900 may be implemented in a number of different forms, as shown in the figure. For example, it may be implemented as a standard server 920, or multiple times in a group of such servers. It may also be implemented as part of a rack server system 924. In addition, it may be implemented in a personal computer such as a laptop computer 922. Alternatively, components from computing device 900 may be combined with other components in a mobile device (not shown), such as device 950. Each of such devices may contain one or more of computing device 900, 950, and an entire system may be made up of multiple computing devices 900, 950 communicating with each other.

Computing device 950 includes a processor 952, memory 964, an input/output device such as a display 954, a communication interface 966, and a transceiver 968, among other components. The device 950 may also be provided with a storage device, such as a Microdrive, solid-state memory or other device, to provide additional storage. Each of the components computing device 950, processor 952, memory 964, display 954, communication interface 966, and transceiver 968 are interconnected using various busses, and several of the components may be mounted on a common motherboard or in other manners as appropriate.

The processor 952 can execute instructions within the computing device 950, including instructions stored in the memory 964. The processor may be implemented as a chipset of chips that include separate and multiple analog and digital processors. The processor may provide, for example, for coordination of the other components of the device 950, such as control of user interfaces, applications run by device 950, and wireless communication by device 950.

Processor 952 may communicate with a user through control interface 958 and display interface 956 coupled to a display 954. The display 954 may be, for example, a TFT LCD (Thin-Film-Transistor Liquid Crystal Display) or an OLED (Organic Light Emitting Diode) display, or other appropriate display technology. The display interface 956 may comprise appropriate circuitry for driving the display 954 to present graphical and other information to a user. The control interface 958 may receive commands from a user and convert them for submission to the processor 952. In addition, an external interface 962 may be provided in communication with processor 952, so as to enable near area communication of device 950 with other devices. External interface 962 may provide, for example, for wired communication in some implementations, or for wireless communication in other implementations, and multiple interfaces may also be used.

The memory 964 stores information within the computing device 950. The memory 964 can be implemented as one or more of a computer-readable medium or media, a volatile memory unit or units, or a non-volatile memory unit or units. Expansion memory 974 may also be provided and connected to device 950 through expansion interface 972, which may include, for example, a SIMM (Single In Line Memory Module) card interface. Such expansion memory 974 may provide extra storage space for device 950, or may also store applications or other information for device 950. Specifically, expansion memory 974 may include instructions to carry out or supplement the processes described above, and may include secure information also. Thus, for example, expansion memory 974 may be provide as a security module for device 950, and may be programmed with instructions that permit secure use of device 950. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 964, expansion memory 974, memory on processor 952, or a propagated signal that may be received, for example, over transceiver 968 or external interface 962.

Device 950 may communicate wirelessly through communication interface 966, which may include digital signal processing circuitry where necessary. Communication interface 966 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 968. In addition, short-range communication may occur, such as using a Bluetooth, Wi-Fi, or other such transceiver (not shown). In addition, GPS (Global Positioning System) receiver module 970 may provide additional navigation- and location-related wireless data to device 950, which may be used as appropriate by applications running on device 950.

Device 950 may also communicate audibly using audio codec 960, which may receive spoken information from a user and convert it to usable digital information. Audio codec 960 may likewise generate audible sound for a user, such as through a speaker, e.g., in a handset of device 950. Such sound may include sound from voice telephone calls, may include recorded sound (e.g., voice messages, music files, etc.) and may also include sound generated by applications operating on device 950.

The computing device 950 may be implemented in a number of different forms, as shown in the figure. For example, it may be implemented as a cellular telephone 980. It may also be implemented as part of a smartphone 982, personal digital assistant, a tablet computer 983 or other similar mobile computing device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

For the sake of clarity, the processes and methods herein have been illustrated with a specific flow, but it should be understood that other sequences may be possible and that some may be performed in parallel, without departing from the spirit of the invention. Additionally, steps may be subdivided or combined. As disclosed herein, software written in accordance with the present invention may be stored in some form of computer-readable medium, such as memory or CD-ROM, or transmitted over a network, and executed by a processor.

All references cited herein are intended to be incorporated by reference. Although the present invention has been described above in terms of specific embodiments, it is anticipated that alterations and modifications to this invention will no doubt become apparent to those skilled in the art and may be practiced within the scope and equivalents of the appended claims. More than one computer may be used, such as by using multiple computers in a parallel or load-sharing arrangement or distributing tasks across multiple computers such that, as a whole, they perform the functions of the components identified herein; i.e. they take the place of a single computer. Various functions described above may be performed by a single process or groups of processes, on a single computer or distributed over several computers. Processes may invoke other processes to handle certain tasks. A single storage device may be used, or several may be used to take the place of a single storage device. The present embodiments are to be considered as illustrative and not restrictive, and the invention is not to be limited to the details given herein. It is therefore intended that the disclosure and following claims be interpreted as covering all such alterations and modifications as fall within the true spirit and scope of the invention.

What is claimed is:

1. A digital aroma system comprising:
a processor;
a motion sensor coupled to the processor for detecting the movements of a vehicle;
a fragrance module comprising an anti-nausea dry fragrance cartridge each having a cartridge housing and a plurality of substrates infused with anti-nausea dry fragrance molecules within the cartridge housing;
an air flow control system controlled by the processor for transmitting air through the anti-nausea dry fragrance cartridge;
a fan or a pump for moving air through the digital aroma system;
wherein the movement signals representing the movement of the vehicle are transmitted from the motion sensor to the processor and when the movement signals are greater than a threshold value the processor opens a first valve to create a first flow path through the anti-nausea dry fragrance cartridge and the air is directed through the anti-nausea dry fragrance cartridge so some of the dry anti-motion sickness fragrance is transmitted from the anti-nausea dry fragrance cartridge to an interior of the vehicle.

2. The digital aroma system of claim 1 further comprising:
a user interface coupled to the processor having an input that is actuated to cause the digital aroma system to emit the anti-motion sickness fragrance.

3. The digital aroma system of claim 2 wherein the user interface is displayed on a mobile computing device running a mobile application program.

4. The digital aroma system of claim 1 further comprising:
a user interface in communication with the processor, the user interface having a nausea level input for inputting a user's level of nausea wherein a quantity of the anti-motion sickness fragrance transmitted from the anti-nausea dry fragrance cartridge by the processor is proportional to the user's level of nausea.

5. The digital aroma system of claim 4 wherein the nausea level input has a low nausea level input and a high nausea level input wherein a lower quantity of the anti-motion sickness fragrance is transmitted from the anti-nausea dry fragrance cartridge by the processor when the low nausea level input is actuated and a higher quantity of the anti-motion sickness fragrance is transmitted from the anti-nausea dry fragrance cartridge by the processor when the high nausea level input is actuated.

6. The digital aroma system of claim 4 wherein the fragrance module includes a plurality of substrates infused with dry fragrance molecules that are not anti-nausea dry fragrance molecules.

7. A digital aroma system comprising:
a processor;
a global positioning system (GPS) coupled to the processor for identifying a position and a velocity of a vehicle;
a mapping system coupled to the processor for identifying a route that the vehicle is traveling on;
a motion sensor coupled to the processor for detecting the movements of the vehicle;
a fragrance module comprising a dry fragrance cartridge having a cartridge housing and a plurality of substrates infused with anti-nausea dry fragrance molecules within the cartridge housing;
an air flow control system with valves controlled by the processor for transmitting air through the dry fragrance cartridge; and
a fan or a pump for moving air through the digital aroma system;
wherein the processor determines that centripetal acceleration forces on the vehicle due to a predicted velocity and curvatures of the road for a predetermined period of time exceed a predetermined value the processor directs the air through the cartridge so some of the dry nausea dry fragrance molecules are transmitted from the cartridge to an interior of the vehicle.

8. The digital aroma system of claim 7 further comprising:
a user interface coupled to the processor having an input that is actuated to cause the digital aroma system to emit the anti-motion sickness fragrance.

9. The digital aroma system of claim 8 wherein the user interface is displayed on a mobile computing device running a mobile application program.

10. The digital aroma system of claim 7 further comprising:
a user database storing nausea locations for a plurality of users wherein when the vehicle is detected to be at one of the nausea locations the processor opens a first valve to create a first flow path through the first cartridge containing an anti-motion sickness fragrance and the air is directed through the first cartridge so some of the dry anti-motion sickness fragrance is transmitted from the first cartridge to the interior of the vehicle.

11. The digital aroma system of claim 7 further comprising:
a user interface in communication with the processor, the user interface having a nausea level input for inputting a user's level of nausea wherein a quantity of the anti-motion sickness fragrance transmitted from the cartridge by the processor is proportional to the user's level of nausea; and
a user database storing nausea locations for a plurality of users wherein when the vehicle is detected to be at one of the nausea locations the processor opens a first valve to create a first flow path through the first cartridge containing an anti-motion sickness fragrance and the air is directed through the first cartridge so some of the dry anti-motion sickness fragrance is transmitted from the first cartridge to the interior of the vehicle.

12. The digital aroma system of claim 7 further comprising:
a user interface in communication with the processor, the user interface having a nausea level input for inputting a user's level of nausea wherein a lower quantity of the anti-motion sickness fragrance is transmitted from the cartridge by the processor when a low nausea level input is actuated and a higher quantity of the anti-motion sickness fragrance is transmitted from the cartridge by the processor when the high nausea level input is actuated.

13. A digital aroma system comprising:
a processor;
a global positioning system (GPS) coupled to the processor for identifying a position and a velocity of a vehicle;
a mapping system coupled to the processor for identifying roads that the vehicle is traveling on;
a motion sensor coupled to the processor for detecting the movements of the vehicle;
a fragrance module comprising a plurality of dry fragrance cartridges each having a cartridge housing and a plurality of substrates infused with fragrance molecules within the cartridge housing;
an air flow control system with valves controlled by the processor for selectively transmitting air through the plurality of dry fragrance cartridges wherein each of the plurality of dry fragrance cartridges has a different fragrance;
a fan or a pump for moving air through the digital aroma system; and
a database in communication with the processor wherein the database stores user nausea locations for a plurality of users;
wherein the processor determines that the vehicle is traveling over at least one of the user nausea locations or at least one of the nausea locations for users in the nausea group and the processor opens a first valve to create a first flow path through a first cartridge containing an anti-motion sickness fragrance and the air is directed through the first cartridge so some of the dry anti-motion sickness fragrance is transmitted from the first cartridge to an interior of the vehicle.

14. The digital aroma system of claim 13 further comprising:
a user interface coupled to the processor having an input that is actuated to cause the digital aroma system to emit the anti-motion sickness fragrance.

15. The digital aroma system of claim 14 wherein the user interface is displayed on a mobile computing device running a mobile application program.

16. The digital aroma system of claim 13 further comprising:
a user database storing nausea locations for a plurality of users wherein when the vehicle is detected to be at one of the nausea locations the processor opens a first valve to create a first flow path through the first cartridge containing an anti-motion sickness fragrance and the air is directed through the first cartridge so some of the dry anti-motion sickness fragrance is transmitted from the first cartridge to the interior of the vehicle.

17. The digital aroma system of claim 13 further comprising:
a user interface in communication with the processor, the user interface having a nausea level input for inputting a user's level of nausea wherein a quantity of the anti-motion sickness fragrance transmitted from the first cartridge by the processor is proportional to the user's level of nausea.

18. The digital aroma system of claim 13 further comprising:
a light controller coupled to lights within the vehicle wherein the light controller adjusts the lights within the vehicle to reduce the user's level of nausea.
